(19) United States
(12) Patent Application Publication
(10) Pub. No.: US 2011/0098826 A1
(43) Pub. Date: Apr. 28, 2011

(54) Disc-Like Angle-Ply Structures for Intervertebral Disc Tissue Engineering and Replacement

(75) Inventors: Robert L. Mauck, Philadelphia, PA (US); Dawn M. Elliott, Philadelphia, PA (US); Nandan Nerurkar, Philadelhia, PA (US)

(73) Assignee: THE TRUSTEES OF THE UNIVERSITY OF PENNSYLVANIA, Philadelphia, PA (US)

(21) Appl. No.: 12/911,166

(22) Filed: Oct. 25, 2010

Related U.S. Application Data
(60) Provisional application No. 61/255,542, filed on Oct. 28, 2009.

(51) Int. Cl. A61F 2/02; A61B 19/02; D04H 3/16

(52) U.S. Cl. 623/23.72; 206/438; 156/167

(57) ABSTRACT

Provided are implant scaffolds comprising angle-ply arrays of two or more layers of substantially aligned fiber, methods of making and using said scaffolds, and kits comprising such scaffolds.

### CROSS REFERENCE TO RELATED APPLICATIONS

This patent application claims the benefit of U.S. Provisional Patent Application Ser. No. 61/255,542 filed Oct. 28, 2009, which is incorporated by reference in its entirety.

### GOVERNMENT SUPPORT

The research carried out in this application was supported, in part, by a grant from the National Institute of Health (National Cancer Institute) through grant R01 EB00245. Pursuant to 35 U.S.C. §202, the government may have rights in any patent issuing from this application.

### TECHNICAL FIELD

The present invention relates to implant scaffolds comprising angle-ply arrays of two or more layers of substantially aligned fiber, methods of making and using said scaffolds, and kits comprising such scaffolds.

### BACKGROUND

The intervertebral disc is a multi-component soft tissue comprising the annulus fibrosus (AF), a multi-lamellar fibrocartilage, and the nucleus pulposus (NP), which together forms the soft tissue structure that transmits loads between vertebrae and permits motion of the spine. It is a complex mechanical system that is subjected to high loads in multiple directions under the activities of daily living. Given its central location and function, and the magnitude of stresses seen with load-bearing use, it is not surprising that damage and/or degeneration of the weakest portion of the disc is a common occurrence, afflicting upward of 97% of the population by 50 years of age. During degeneration, the AF becomes progressively disorganized, concomitant with mechanical and structural failure including tears, fissures, and delamination, each of which is thought to contribute to low back pain. At the same time, the soft, hydrated NP progressively becomes stiffer and more fibrous. Treatments for discogenic back pain and disc degeneration are largely palliative, and restoration of function remains unaddressed. Current surgical treatments such as discectomy, fusion, and total disc arthroplasty may alleviate the pain, but fail to restore the function to the disc and may lack long term efficacy. Therefore, there is great need for regenerative strategies that may alleviate low back pain while restoring function and range of motion to the spine. While synthetic implants are efficacious and have a long history of use for knee and hip replacement, similar products have only recently been introduced for disc replacement, and their long term efficacy has yet to be established.

To date, numerous strategies have been proposed to engineer replacement tissues for the annulus fibrosus and the intervertebral disc. See, e.g., R. A. Kandel, S. Roberts, and J. Urban, Eur Spine J 17, 5480 (2008). However, these studies have failed to appreciate the angle-ply microstructure that is necessary for proper mechanical function of the native tissue. See, e.g., Mauck, R. L., et al., “Engineering on the Straight and Narrow: The Mechanics of Nanofibrous Assemblies for Fiber-Reinforced Tissue Regeneration,” Tissue Eng., Part B Rev., 2009 Feb. 10; Nerurkar, N. L., et al., “Mechanics of oriented electrospun nanofibrous scaffolds for annulus fibrosus tissue engineering,” J. Orthop. Res., 2007 August; 25(8):1018-1028; Yang, L., et al., “Polar surface chemistry of nanofibrous polyurethane scaffold affects annulus fibrosus cell attachment and early matrix accumulation,” J. Biomed. Mater. Res. A, 2008 Dec. 23; Gruber, H. E., et al., “Culture of human annulus fibrosus cells on polyamide nanofibers: extracellular matrix production,” Spine, 2009 Jan. 1, 34(1): 4-9; Nesti, et al., “Intervertebral disc tissue engineering using a novel hyaluronic acid-nanofibrous scaffold (HANFS) amalgam,” Tissue Eng. Part A, 2008 September; 14 (9), 1527-1 537; Nerurkar, N. L., et al., “ISSLS prize winner: Integrating theoretical and experimental methods for functional tissue engineering of the annulus fibrosus,” Spine, 2008 Dec. 1, 33(25): 2691-2701; Baker B. M., et al., “The potential to improve cell infiltration in composite fiber-aligned electrospun scaffolds by the selective removal of sacrificial fibers,” Biomaterials, 2008 May, 29(15):2348-2358; Baker, B. M., et al., “Multi-Lamellar and Multi-Axial Maturation of Cell-Seeded Fiber-Reinforced Tissue Engineered Constructs,” Proceedings of ASME 2007 Summer Bioengineering Conference, Keystone, Colo., Jun. 20-24, 2007; Mizuno, et al., “Biomechanical and biochemical characterization of composite tissue-engineered intervertebral discs,” Biomaterials 27 (3), 362-370 (2006); Shao and Hunter, “Developing an alginate/chitosan hybrid fiber scaffold for annulus fibrosis cells,” J. Biomed. Mater. Res. A. (82) 710-710 (2007). Despite efforts in this area, it remains a challenge to engineer a multilamellar AF with aligned, opposing fiber orientations similar to the native AF. If anything, these studies have shown that, in the absence of a scaffold that provides the appropriate structural—and perhaps mechanical—cues, cells are unable to spontaneously organize their extracellular matrix into highly specialized architectures like that of the annulus fibrosus. The function of the annulus fibrosus is predicated on a high degree of structural organization over multiple length scales: aligned bundles of collagen fibers reside within each lamella and the direction of alignment alternates from one lamella to the next. The resulting angle-ply laminate possesses pronounced mechanical anisotropy and nonlinearity. Consequently, no engineered tissue has successfully achieved mechanical properties that are commensurate with the native tissue.

### SUMMARY

In the face of these shortfalls in the existing art, the present invention describes an implant scaffold comprising at least two overlapping layers, each layer comprising at least one fiber aligned along a major axis of said layer, the layers being positioned such that the major axis of a first layer forms an oblique angle with respect to the major axis of a second layer, said oblique angle defining a long axis within the arc of the oblique angle. Such constructions provide for scaffolds comprising two overlapping layers of substantially aligned fiber, or multilayer constructs.

Such angle-ply arrays provide embodied scaffolds with improved shear and torsional stability, as well as improved tensile modulus. Additional embodiments provide that, when more than two layers are present, the major axis of subsequent layers may be substantially parallel with either of these first two layers, or may be positioned so as to be oblique to both. Still other embodiments of this invention teach that, in such multilayer constructs, the degree of orientation or composition of the fibers vary across layers.

In addition to structural organization within individual layers, certain embodiments also provide for connectivity between layers. These embodiments provide that at least one fiber from at least one layer is chemically or physically joined to at least one fiber in at least one other layer. This interconnectivity between layers provides for additional structural integrity and performance enhancement, and can be accomplished by chemical crosslinking, through the use of adhesives, heat, pressure, microwave radiation, or combinations thereof, or through the use of growing tissue.

Other embodiments of the present invention describe the scaffold in terms of its physical characteristics. For example, the invention provides that the scaffold modulus, when measured along the long axis, is greater than the modulus of any individual layer, when measured along the same directional axis. In certain embodiments, the modulus, when measured along the long axis, is at least about 30% greater, preferable at least about 40%, more preferably at least about 50%, and even more preferably at least about 70% than the modulus of any individual layer, when measured along the same directional axis.

In absolute value terms, certain embodiments provide that the scaffold modulus, or layers therefrom, when measured along the long axis, is at least 6 MPa, preferably at least 8 MPa, more preferably at least 12 MPa, still more preferably at least 14 MPa, still more preferably at least 16 MPa, and still more preferably at least 18 MPa. This invention also teaches the ability to provide moduli of scaffolds which mimic those of the biologic to be replaced. For example, for those scaffolds designed to replace an annulus fibrosus (AF), one preferred embodiment describes a multilayer scaffold in which the layers corresponding to the inner AF exhibit a modulus of about 6-8 MPa, whereas the layers corresponding to the outer AF exhibit a modulus of about 15-20 MPa, both when measured along the long axis.

In other embodiments, the torsional response is non-linear. Moreover, the angle-ply arrangement of the scaffold provides substantially higher values than that provided by any individual layer.

Another distinguishing feature of this invention is that the scaffold can be substantially anatomically shaped, non-limiting examples including the substantial shape of an annulus fibrosus or a knee meniscus, and that the scaffold can be formed in such a substantial shape. That is, certain embodiments provide for planar structures, non-planar conformal surfaces and those wherein the long axis is circumferential to a center-line axis. Such latter embodiments may describe a scaffold comprising a layered ring structure in which the individual layers are oriented radially from a center line axis, and the long axis is circumferential to this same center line axis. In addition to fully circular or quasi-circular structures, other embodiments also provide that this spatial conformation be maintained or provided in arc segments of the circular or quasi-circular structure.

The invention is also flexible in the choice of materials. Cross-sections of the fiber or fibers may be circular, oval, rectangular, square, or any shape which can be defined, for example, by a spinneret. Similarly, the fibers can have thickness dimensions in the range of about 1 nm to about 10 microns.

As described herein, various embodiments describe that fibers may comprise materials which are natural, synthetic, biocompatible, biodegradable, non-biodegradable, and/or biosorbable. In some embodiments, at least one layer contains a fiber comprising a porogen or a fiber which is photolytically active. The invention also describes that more than one polymer may be used to fabricate the individual layers of the present invention; for example, each layer may be fabricated from multiple co-spun polymer or co-polymers, or from a mixture of simultaneously or sequentially delivered polymers and/or copolymers.

Other embodiments of this invention provide that at least one fiber is biodegradable in a physiological fluid, said fluids including water, saline, simulated body fluid, or synovial fluid. Where the scaffold comprises two or more biodegradable fibers, each can have a different biodegradation and/or biosorption profile. In certain embodiments, the biodegradation and/or biosorption profile of the at least one biodegradable fiber is chosen to approximately coincide with the rate of ingression of tissue growth. In this way, the degradation in modulus of the scaffold can be made to match or partially offset the temporal stiffening associated with ingression of the growing tissue, thus allowing a system to be designed with approximately constant temporal performance parameters.

In addition to the fibers, the scaffold can also comprise a variety of additional materials, added before (e.g., during formation of the fiber), during (e.g., between individual layers) or after the formation of the scaffold laminate. These materials may be applied uniformly throughout the scaffold or so as to provide gradients across or through the scaffold.

In one such case, certain embodiments provide that the scaffold comprises at least one gel. Other embodiments describe these additional materials as comprising biofactors, therapeutic agents, particles, or cells. The invention further provides that these biofactors, therapeutic agents, particles, or other materials incorporated within the scaffold, as required or desired, may be biodegraded, dissolved, and/or released according to a predetermined time profile. In other embodiments, these changes occur so as to complement the entry and incorporation of cells and/or tissues within the scaffold.

Another embodiment provides that the scaffold further comprises at least one population of cells. These populations of cells can exist as homogeneous or heterogeneous mixtures within or across at least one layer or at the interface of two individual layers, or provide a scaffold having at least one gradient across the various dimensions of the scaffold. These gradients can be continuous or step-wise, as with the other components, as determined by the processing parameters. In other embodiments, these cells develop into tissue, such that the scaffolds comprise growing tissue corresponding to the cells used.

The invention also describes embodiments directed toward making the scaffolds heretofore described. Particular embodiments include a method of making an implant scaffold comprising contacting at least two layers, each layer comprising at least one fiber aligned along a major axis of said layer, the layers being positioned such that the major axis of a first layer forms an oblique angle with respect to the major axis of a second layer, said oblique angle defining a long axis within the arc of the oblique angle.

In forming the scaffold from individual layers, several methodologies are contemplated by this invention. Exemplary examples include embodiments wherein each layer is individually formed and physically isolated, either by electrospinning or any other method which provides for layers of substantially aligned fibers as described herein. These physically isolated layers are then apositioned such that the major axis of layer is oriented to be consistent with the descriptions provided above for the scaffolds. In other embodiments, an individual layer or combined layers are physically isolated and additional layers are applied using direct electrospinning onto the layer or layers.

To form more complicated anatomical constructs using these layered scaffolds, one embodiment provides that two or more layers are wound around a cylindrical form, such that the long axis is made to be circumferential to the center-line axis of the cylindrical mold. Other embodiments provide for non-cylindrical molds. Similarly, conformance of layered scaffold laminates to non-planar molds, or around non-planar objects may be used to provide desired constructs.

Similarly, additional embodiments provide that the individual layers be joined, either physically or chemically, or by growing tissue within the scaffold or by the application and thickening of gel materials.

In still other embodiments, where at least one layer contains a porogenic material, that material may be removed before the application of at least one population of cells, and/or the application of at least one therapeutic agent, biofactor, catalyst, or mixture or combination thereof.

Still other embodiments provide that tissue be grown on or within the scaffold. This may be done in vitro, in vivo, or in a process combining the two methods.

This invention also provides for one or more kits containing a packaged sterilized implant scaffold, in which these kits comprise any of the various embodiments, including one or more of the properties and characteristics described herein, and may include at least one plate for connecting to and/or distributing the forces of the neighboring bone over the surface of the implant, a carrier for the scaffold, an insertion adapter, such as a head, holder, or other carrier, and/or a jacket surrounding the scaffold, the jacket constraining hydration of the scaffold to the partial or fully hydrated state.

The invention further provides for embodiments describing the use of these implant scaffolds in patients. One such embodiment provides a method of treating a mammalian patient comprising: (a) assessing the need to repair or replace at least one body part of said patient; (b) deciding that implanting a scaffold to facilitate the repair or replacement of said body part is a viable treatment for said patient; and (c) implanting into said patient an implant scaffold comprising at least two overlapping layers, each layer comprising at least one fiber aligned along a major axis of said layer, the layers being positioned such that the major axis of a first layer forms an oblique angle with respect to the major axis of a second layer, said oblique angle defining a long axis within the arc of the oblique angle. The invention further provides that these substantially anatomically shaped solids can be used as scaffold implants, to replace or repair their corresponding anatomical part. Such parts include cartilage (including, elastic, hyaline, and fibrocartilage), collagen, adipose tissue, reticular connective tissue, embryonic connective tissues (including mesenchymal connective tissue and mucous connective tissue), tendons, ligaments, and bone, blood vessels, corneas, rotator cuff tendons, urinary bladder walls, diaphragms, and other biologic orthopedic or cardiovascular laminates which would benefit from the angle-ply structures of the present invention.

### BRIEF DESCRIPTION OF THE DRAWINGS

The summary, as well as the following detailed description, is further understood when read in conjunction with the appended drawings. For the purpose of illustrating the invention, there are shown in the drawings exemplary embodiments of the invention; however, the invention is not limited to the specific methods, compositions, and devices disclosed. In addition, the drawings are not necessarily drawn to scale. In the drawings:

FIG. 1 shows various orientations of electrospun fiber. FIG. 1A shows a random orientation of fibers; FIGS. 1B through 1D show arrays of substantially oriented fibers. The arrows shown in FIGS. 1C and 1D show the major axis for the fiber sets as described herein.

FIG. 2 A shows schematic representations of parallel, oblique, and perpendicular sets of fibers. FIG. 2B is a schematic representation of the concept of the oblique angle and that of the long axis, as described herein.

FIG. 3 shows a schematic representation of a scaffold of the present invention comprising a layered ring structure in which the individual layers are oriented radially from a center line axis, and the long axis is circumferential to this same center line axis.

FIG. 4 shows an example of a mold which may be used to construct the scaffold of FIG. 3.

FIG. 5 illustrates what is meant when it is written that scaffolds were excised 30° from the prevailing fiber direction of electrospun nanofibrous mats to replicate the oblique collagen orientation within a single lamella of the annulus fibrosus (FIG. 5A). At 0 weeks, MSC seeded scaffolds were formed into bilayers between pieces of porous polypropylene and wrapped with a foil sleeve (FIG. 5B). Bilayers were oriented with either Parallel) (+30°/+30°) or Opposing) (+30°/−30°) fiber alignment relative to the long axis of the scaffold. PP=porous polypropylene; F=foil.

FIG. 6 graphically depicts the data gathered when the extracellular matrix within bilayers seeded with mesenchymal stem cells in Example 4. Sulphated glycosaminoglycan (s-GAG, FIG. 6a) and collagen (FIG. 6b) content of Parallel and Opposing bilayers increased with culture duration (p≦0.05). There were no significant differences between Parallel and Opposing bilayers at any time point. Alcian Blue (FIG. 6c), Picrosirius Red (FIG. 6d), and DAPI (FIG. 6e) staining of Opposing bilayer cross-sections after 10 weeks of in vitro culture. DW=dry weight. Dashed line indicates content at 0 weeks, when bilayers were formed. Error bars (a, b) represent the standard deviation of the mean. * indicates inter-lamellar space. Scale bar=250 pm (c, d), 200 pm (e). Scale bar in FIG. 6c is 200 microns; in FIGS. 6d and e is 250 microns.

FIG. 7 depicts the angle-ply collagen alignment and orientation described in Example 5. Sections were collected obliquely across lamellae (FIG. 7a), stained with Picrosirius Red, and viewed under polarized light microscopy to visualize collagen organization. When viewed under crossed polarizers, birefringent intensity indicates the degree of alignment of the specimen, while the hue of birefringence indicates the direction of alignment. After 10 weeks of in vitro culture, Parallel bilayers contained co-aligned intra-lamellar collagen within each lamella (FIG. 7b). Opposing bilayers contained intra-lamellar collagen aligned along two opposing directions (FIG. 7c), successfully replicating the gross fiber orientation of native bovine annulus fibrosus (FIG. 7d). In engineered bilayers, as well as the native annulus fibrosus, a thin layer of disorganized (nonbirefiingent) collagen was observed at the lamellar interface (denoted by *). The distribution of collagen fiber orientations was determined by quantitative polarized light analysis. Prominent peaks in fiber alignment were observed near 30° in both lamellae of Parallel bilayers (FIG. 7e); however in Opposing bilayers two fiber populations were observed, aligned along +30° and −30° (FIG. 7f). Scale bar=200 pm (FIG. 7b, c), 100 pm (FIG. 7d). L112=Lamella 112; IL=Inter-lamellar space.

FIG. 8 graphically depicts the data obtained from Example 6, relating inter-lamellar mechanics with the tensile response of biologic laminates. Uniaxial tensile moduli of MSC-seeded Parallel and Opposing bilayers increased with in vitro culture duration, with Opposing bilayers achieving significantly higher moduli than Parallel bilayers from 4 weeks onward (FIG. 8a). #=p≦0.05 compared to single lamellar modulus at 0 weeks. +=p≦0.05 compared to Parallel modulus. Native=circumferential tensile modulus of native human AF. Lap testing of MSC-seeded laminates showed increasing interface strength with in vitro culture duration (FIG. 8b). #=p≦0.05 compared to 2 weeks. To elucidate the role of interface properties on the tensile response of bilayers, uniaxial tensile testing was performed on acellular bilayers formed from nanofibrous scaffolds bonded together by agarose of increasing concentrations (FIG. 8c). Increasing inter-lamellar agarose concentration—and hence inter-lamellar stiffness—significantly increased the tensile modulus of acellular Opposing bilayers, but had no effect on the Parallel bilayer group. #=p≦0.05 compared to orientation-matched 2% agarose. +=p≦0.05 compared to concentration-matched Parallel bilayers. All error bars (FIG. 8a-c) represent standard deviations of the mean.

FIG. 9A is a schematic showing of fabrication process for formation of engineered disc-like angle-ply structure (DAPS) (NFS: nanofibrous scaffold). FIG. 9B illustrates the gross morphology of a scaffold prepared with nanofibrous AF region and agarose NP region, scale bar: 1 mm. FIG. 9C provides a close up view of AF region enlarged from box in FIG. 9B. Representative stress-relaxation (FIG. 9D) and torsion (FIG. 9E) response of DAPS showing the viscoelastic and non-linear response of the composite.

FIG. 10A is an SEM of AF region after 1 week of culture. FIG. 10B provides a higher magnification SEM of interface formation between individual lamellae at 1 week time point. Actin and DAPI staining of cells (FIG. 10C) and Picrosirius Red staining of newly formed collagen (FIG. 10D) organized in alternating directions along interface within sections taken oblique to the axial plane. Scale bar in each case: 250 microns.

FIG. 11A shows a DAPI staining of transverse section of DAPS at 1 week, as described in Example 14, showing homogenous distribution of MSCs in the ‘NP’ region, and lamellar organization of MSCs in the ‘AF1 region. Scale: 500 microns. Note: separation of NP and AF occurred as an artifact of sectioning. FIG. 11B is a polarized light image of Picrosirius Red stained oblique section of ‘AF’ region at 6 weeks showing birefringent material in opposing orientations with progression through adjacent lamellae. Scale: 250 microns.

FIG. 12A shows Alcian Blue staining and FIG. 12A shows Picrosirius Red staining of 6 week constructs described in Example 14 with magnified images from NP and AF regions shown as indicated. Scale=500 microns (FIGS. 12A, B).

FIG. 13A shows a contrast profiles of the scaffold described in Example 15. Collagen (FIG. 13B), GAG (FIG. 13C), and DNA (FIG. 13D) content, reported in % wet weight (% wt/wt) for the ‘NP’ and ‘AF’ regions as a function of time in culture. Solid and dashed lines indicate native lapine AF and NP benchmarks, respectively. ‘indicates p≦0.05 for compared to time-matched ‘NP’ values; * indicates p≦0.05 for compared to 1 week time point. Results presented as the mean+SD for 4 samples/group per time point. Scale bar: 250 microns.

FIG. 14 graphically illustrates the equilibrium modulus (FIG. 14A) and percent stress relaxation (FIG. 14B) measured by unconfined compression for the DAPS as a function of time. # indicates p≦0.05 compared to the 1 week time point. Results presented as the mean+SD for 4 samples/group per time point.

### DETAILED DESCRIPTION OF ILLUSTRATIVE EMBODIMENTS

The present invention may be understood more readily by reference to the following detailed description taken in connection with the accompanying Figures and Examples, which form a part of this disclosure. It is to be understood that this invention is not limited to the specific products, methods, conditions or parameters described and/or shown herein, and that the terminology used herein is for the purpose of describing particular embodiments by way of example only and is not intended to be limiting of any claimed invention. Similarly, any description as to a possible mechanism or mode of action or reason for improvement is meant to be illustrative only, and the invention herein is not to be constrained by the correctness or incorrectness of any such suggested mechanism or mode of action or reason for improvement. Throughout this text, it is recognized that the descriptions refer both to the method of preparing such devices and to the resulting, corresponding physical devices themselves, as well as the referenced and readily apparent applications for such devices.

In the present disclosure the singular forms “a,” “an,” and “the” include the plural reference, and reference to a particular numerical value includes at least that particular value, unless the context clearly indicates otherwise. Thus, for example, a reference to “a material” is a reference to at least one of such materials and equivalents thereof known to those skilled in the art, and so forth.

When values are expressed as approximations, by use of the antecedent “about,” it will be understood that the particular value forms another embodiment. In general, use of the term “about” indicates approximations that can vary depending on the desired properties sought to be obtained by the disclosed subject matter and is to be interpreted in the specific context in which it is used, based on its function, and the person skilled in the art will be able to interpret it as such. Where present, all ranges are inclusive and combinable.

It is to be appreciated that certain features of the invention which are, for clarity, described herein in the context of separate embodiments, may also be provided in combination in a single embodiment. Conversely, various features of the invention that are, for brevity, described in the context of a single embodiment, may also be provided separately or in any subcombination. Further, references to values stated in ranges include each and every value within that range.

Generally terms are to be given their plain and ordinary meaning such as understood by those skilled in the art, in the context in which they arise. To avoid any ambiguity, however, several terms are described herein.

The disclosures of each patent, patent application, and publication cited or described in this document are hereby incorporated herein by reference, in their entirety.

The present invention discloses an implant scaffold comprising at least two overlapping layers, each layer comprising at least one fiber aligned along a major axis of said layer, the layers being positioned such that the major axis of a first layer forms an oblique angle with respect to the major axis of a second layer, said oblique angle defining a long axis within the arc of the oblique angle.

The term “fiber” refers to a population of at least one fiber type, each fiber type made of at least one material, generally polymeric, and each fiber type comprising a plurality of fiber strands. That is, a “fiber” does not necessarily refer to a single strand of fiber. For example, when an electrospun solid is made of a single “fiber” (e.g. nanofiber), the fiber is folded thereupon, hence can be viewed as a plurality of connected fiber strands. In certain embodiments, a “fiber body” may comprise one fiber or multiple fiber populations, each fiber comprising fiber strands of two or more different materials. It is to be understood that a more detailed reference to a fiber is not intended to limit the scope of the present invention to such particular case. Thus, unless otherwise defined, for example, by use of the term “strand” or “individual strand,” any reference herein to a “fiber” applies to a plurality of strands of at least one type of fiber. In cases where multiple types of fiber are present, the term “fiber” describes this multiplicity, unless otherwise specified.

Electrospinning is one method, known by those skilled in the art to which this invention pertains, for generating nano- and micron-scale fibers, capable of recapitulating the organizational features and length-scales of many collagenous tissues. In its most basic form, electrospinning involves the application of a high voltage potential and resulting gradient to draw a polymer solution from spinnerets into thin fibers which can then be collected on a surface en masse. While this disclosure describes articles and methods of making using electrospinning, it should be recognized that these articles and methods are not necessarily constrained to this technology.

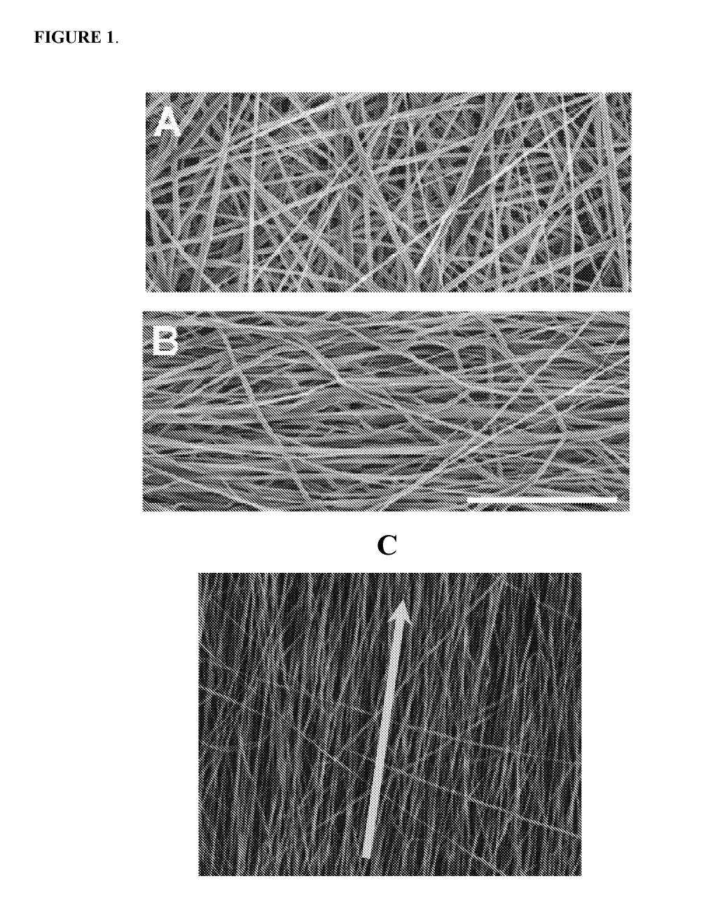

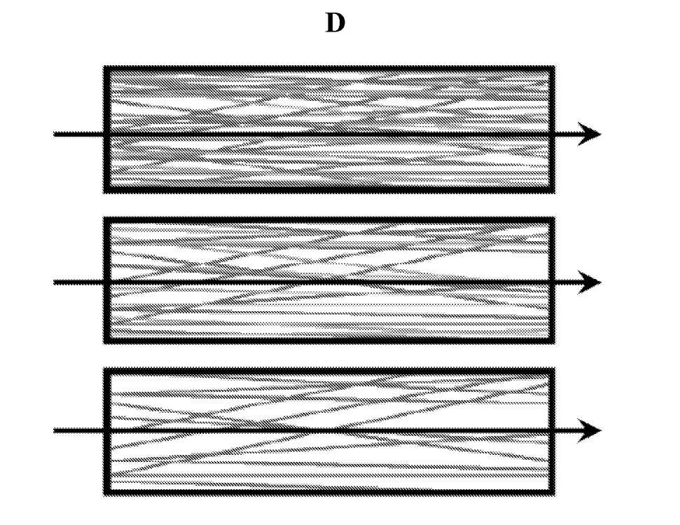

In electrospinning, the degree to which fibers orient or align is a function of processing. For example, fibers spun onto stationary surfaces tend to be randomly distributed, as shown in FIG. 1A. However, by collecting the fibers on a rotating mandrel (or an otherwise moving surface), it is possible to orient or align the electrospun fibers circumferentially to the direction of rotation of the mandrel (or in the direction of the moving surface). That is, any technique capable of providing substantially aligned fibers is embraced by this invention; for example, electrospinning onto a rapidly oscillating collection surfaces may provide for this effect. Such aligned fibers are shown, for example, FIG. 1B. The degree of alignment depends on various parameters, including the rotational speed of the mandrel (surface speed), the speed of transverse movement of the spinneret relative to the rotational speed of the mandrel (surface speed), the distance of the spinneret from the collection surface, and the rate of delivery of the polymer to the surface, which in part depends on the strength of the potential field between the spinneret and the mandrel. The skilled artisan is capable of increasing or decreasing the relative degrees of orientation or alignment of an electrospun article, without undue experimentation.

Directionality is implicit in the concept of alignment or substantial alignment. As used herein, the term “major axis,” as in the “major axis” of an individual layer of fiber, refers to the direction of alignment within that layer. Again referring to FIGS. 1C and 1D, in each case the fibers are “substantially aligned” (in the direction of the arrows) and are characterized as being so aligned to the major axes as indicated.

As used herein, the terms “substantially aligned” or “substantial alignment” refers to a general directional orientation of a fiber or fibers, without necessary regard for the degree of alignment. That is, the term refers to a fiber or fibers generally running in the same direction, or tending to run in the same average direction. It is used to connote anisotropy of fiber orientation and to distinguish from a random orientation. FIGS. 1B through 1D show fibers that are substantially aligned, running in the same direction, despite variations in the degree of orientation of the individual fiber strands. As shown in these FIGURES, it is not necessary that all of the individual strands of fibers run parallel within a layer; in fact, it is common that individual strands depart from parallel, even in highly oriented electrospun fibers. Such departures by individual strands do not detract from the concept of “substantial alignment” or being “substantially aligned.” To be substantially aligned requires only a measurable degree of directionality. One non-limiting way to quantify this degree of alignment is to describe the deviation of individual fiber strands from the major axis. For examples, the fiber strands are aligned if the majority of the strands are within 80° of the major axis. Additionally, fibers within 60°, within 40°, within 20°, or within 10° of the major axis all are encompassed by embodiments of this invention.

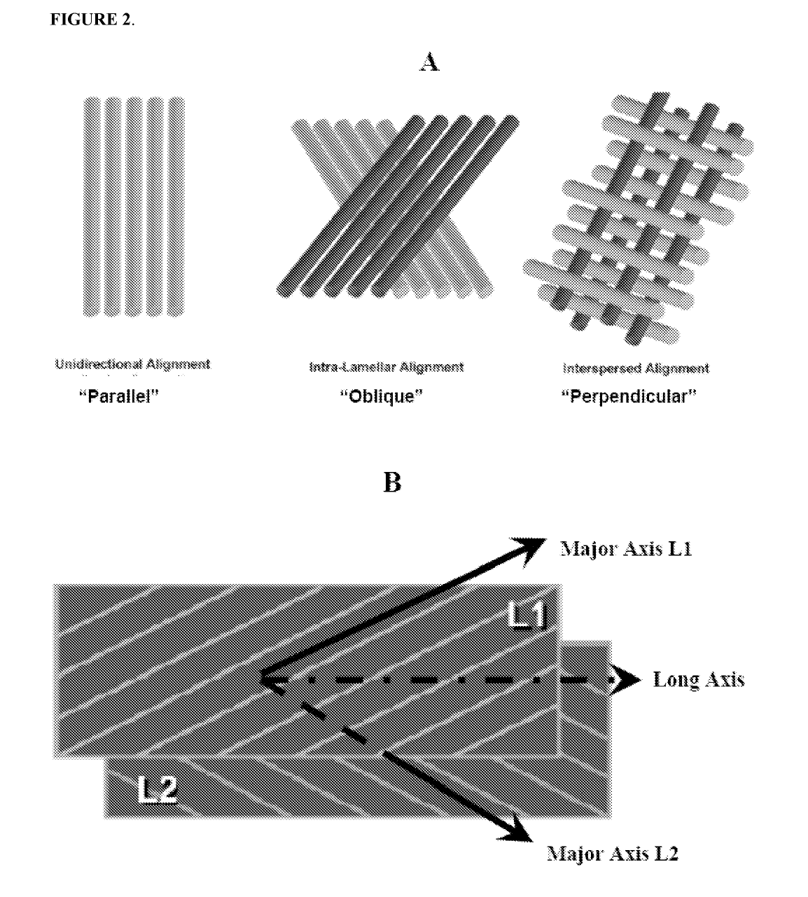

One embodiment of the present invention describes an implant scaffold as comprising two layers of substantially aligned fiber, overlapping so as to be oriented one over the other. Other embodiments provide for more than two layers, in a multilayer construct. In each case, the minimal requirement is that at least two of these layers are arranged in an angle-ply array, such that the major axis of one layer forms an oblique angle with the major axis of another, as shown in FIG. 2A. “Oblique” refers to an angle defined by major axes which is neither parallel nor perpendicular to one another. In certain embodiments, the oblique angle is in the range of about 20° to about 160°, preferably in the range about 40° to about 120°, more preferably in the range about 40° to about 90°, and more preferably in the range about 50° to about 70°.

This angle-ply array provides attractive properties to the scaffold, including shear and torsional stability, as well as improved tensile modulus. In particular, as disclosed herein, the tensile modulus is improved when the scaffold is tested in a direction intermediate to either of the individual major axes (relative to the tensile modulus for either layer if tested in the same direction); that is, in a direction between the two major axes, or at an angle within the arc defined by the oblique angle. As used herein, the term “long axis” is used to describe this intermediate direction, and is shown in FIG. 2B. While it appears generally preferred that the “long axis” actually bisects the oblique angle, such that the angle between the major axis of the first layer and the long axis is the same as the angle between the major axis of the second layer and the long axis, it is not required that the long axis be so defined. Different applications will consider embodiments where the long axis is not bisecting to be important. Among the embodiment of this invention, the angles defined by the major axis of each of the first and second layers with the long axis are independently in the range of about 10° to about 80°, preferably in the range about 20° to about 60°, more preferably in the range about 20° to about 45°, and more preferably in the range about 25° to about 35°. Again, preferred embodiments describe that, while the angles may be independently defined, they may also be the same. In such cases, the relative positioning of the major axes relative to the long axis may be described as “oppositely oriented” or being oriented at “±” some number of degrees. For example, the circumstance where the angles defined by both major axes relative to the long axis are 30° may be described herein as being “oppositely oriented at 30°” or “oriented ±30°” with respect to the long axis.

Additional embodiments provide for these angle-ply scaffolds whose shear or torsional stability are measurably better than otherwise observed for scaffolds otherwise equivalent except for the absence of this angle-ply feature, when tested using methods of the art.

When more than two layers are present, the major axis of subsequent layers may be substantially parallel (or coincident, e.g., within about 10° of one another) with either of these first two layers, or may be positioned so as to be oblique to both. When oblique to both, the invention describes that the angle between the major axis of each additional layer and the long axis is independently in the range of about 10° to about 80°, preferably in the range about 20° to about 60°, more preferably in the range about 20° to about 45°, and more preferably in the range about 25° to about 35°.

Still other embodiments of this invention teach that, in such multilayer constructs, the degree of orientation or composition of the fibers vary across layers. For example, in one non-limiting example of this concept, a first pair of layers may be oriented such that the major axes of these layers are oppositely oriented at 45° with respect to the long axis, whereas a second pair of layers, within the same multilayer construct, is oriented oppositely at 28° with respect to the long axis. This may be done, for example, to replicate a desired set of performance properties.

Other embodiments remove the constraint that every layer be substantially aligned; that it, the scaffold may comprise at least one randomly oriented layer of fiber.

The invention also teaches that at least one layer of the scaffold has a thickness in the range of about 50 micron to about 500 micron thick, preferably in the range of about 100 microns and about 500 microns, more preferably in the range of about 200 microns to about 400 microns, and more preferably about 250 microns. In still other embodiments, every layer has a thickness in the range of about 50 microns to about 500 microns, preferably in the range of about 100 microns and about 500 microns, more preferably in the range of about 200 microns to about 400 microns, and more preferably about 250 microns.

In addition to structural organization within individual layers, certain embodiments also provide for connectivity between layers. These embodiments provide that at least one fiber from at least one layer is chemically or physically joined to at least one fiber in at least one other layer. This interconnectivity between layers provides for additional structural integrity and performance enhancement.

This interconnectivity between layers can be based on chemical or physical attachments and accordingly accomplished chemically or physically. For example, in one set of embodiments, at least one fiber from one layer is chemically crosslinked to at least one fiber in another layer. In other embodiments, the interconnectivity is accomplished through the use of adhesives, heat, pressure, microwave radiation, or combinations thereof, by including fibers susceptible to welding under such conditions in adjacent layers, and then so processing them.

In still other embodiments, growing tissue within the scaffold provides the necessary degree of connectivity, such that at least one fiber in at least one layer is joined by the growing tissue to at least one fiber in at least one other layer.

Other embodiments of the present invention describe the scaffold in terms of its physical characteristics. For example, the invention provides that the scaffold modulus, when measured along the long axis, is greater than the modulus of any individual layer, when measured along the same directional axis. In certain embodiments, the modulus, when measured along the long axis, is at least about 30% greater, preferable at least about 40%, more preferably at least about 50%, and even more preferably at least about 70% than the modulus of any individual layer, when measured along the same directional axis.

In other embodiments, the torsional response is non-linear. Moreover, the angle-ply arrangement of the scaffold provides substantially higher values than that provided by any individual layer.

In absolute value terms, certain embodiments provide that the scaffold modulus, or layers therefrom, when measured along the long axis, is at least 6 MPa, preferably at least 8 MPa, more preferably at least 12 MPa, still more preferably at least 14 MPa, still more preferably at least 16 MPa, and still more preferably at least 18 MPa. This invention also teaches the ability to provide moduli of scaffolds which mimic those of the biologic to be replaced. For example, for those scaffolds designed to replace an annulus fibrosus (AF), one preferred embodiment describes a multilayer scaffold in which the layers corresponding to the inner AF exhibit a modulus of about 6-8 MPa, whereas the layers corresponding to the outer AF exhibit a modulus of about 15-20 MPa, both when measured along the long axis.

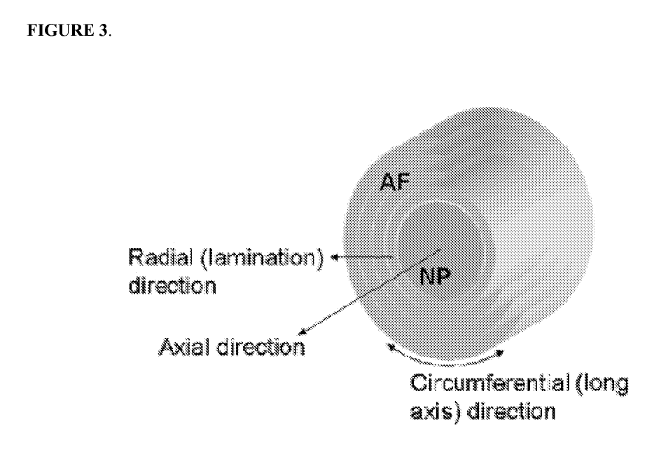

Another distinguishing feature of this invention is that the scaffold can be substantially anatomically shaped, non-limiting examples including the substantial shape of an annulus fibrosus or a knee meniscus, and that the scaffold can be formed in such a substantial shape. While the disclosure has thus far described the implantable scaffold in terms which may suggest a planar structure, the invention is not so limiting, and in certain cases, alternative conformations may be preferred. Certain embodiments describe a scaffold wherein the long axis is circumferential to a center-line axis; that is, these embodiments describe a scaffold comprising a layered ring structure in which the individual layers are oriented radially from a center line axis, and the long axis is circumferential to this same center line axis. Such an arrangement is shown in FIG. 3, and is consistent with a structure actually seen in the annulus fibrosus. Indeed, a scaffold exhibiting the structural and mechanical features of the annulus fibrosus is a preferred embodiment of the present invention Again, such a conformation provides for structures whose compositions and fiber orientations vary with the radial distance from the center-line axis.

In addition to fully circular or quasi-circular structures (e.g., oval or bean-shaped structures, such as that exhibited by intervertebral discs), other embodiments also provide that this spatial conformation be maintained or provided in arc segments of the circular or quasi-circular structure.

Similarly, other embodiments provide from structures wherein the three-dimensional shape of the scaffold includes torsional twists, as well as or instead of circular or quasi-circular arrangements.

Similarly, sheets of scaffold laminates may comprise non-planar shapes, so as to conform to any non-planar surface. Such a non-planar shape may be prepared by conforming an originally planar scaffold laminate to a complementary curved surface.

The invention is also flexible in the choice of materials. Turning to the materials of construction, the invention is not constrained by the thickness or shape of the fibers used, whether generated by electrospinning or otherwise. Accordingly, the cross-sections of the fiber or fibers may be circular, oval, rectangular, square, or any shape which can be defined, for example, by a spinneret. Similarly, the fibers can have thickness dimensions in the range of about 1 nm to about 10 microns, in the range of about 20 nm to about 1000 nm, in the range of about 100 nm to about 1000 nm, or in the range of about 1 micron to about 10 microns, depending on the application. As used herein, the term “nano-scale” refers to dimensions, typically thickness, in the range of about 1 nm to about 1000 nm; similarly, the term “nanofibers” refers to polymer fibers having diameters typically between 10 nm and 1000 nm. Exemplary sub-ranges contemplated by the present invention include between 100 and 1000 nm between 100 and 800 nm, between 100 and 600 nm, and between 100 and 400 nm. Other exemplary ranges include 10-100 nm, 10-200 nm and 10-500 nm. As mentioned, the fibers of the scaffolds of the present invention are preferably generated by an electrospinning process.

As described herein, the various fibers may comprise materials which are natural, synthetic, biocompatible, biodegradable, non-biodegradable, and/or biosorbable. Unless specifically restricted to one or more of these categories, the fibers may comprise materials from any one of these categories. For performance reasons, it may be desirable to incorporate biodegradable or porogenic materials into the design. Further, to be implantable, most embodiments provide that the materials used are at least biocompatible, and preferable approved by the United States Food and Drug Administration in the United States (or a corresponding regulatory agency in other countries).

The phrase “synthetic polymer” refers to polymers that are not found in nature, even if the polymers are made from naturally occurring biomaterials. Examples include, but are not limited to, aliphatic polyesters, poly(amino acids), copoly(ether-esters), polyalkylenes oxalates, polyamides, tyrosine derived polycarbonates, poly(iminocarbonates), polyorthoesters, polyoxaesters, polyamidoesters, polyoxaesters containing amine groups, poly(anhydrides), polyphosphazenes, polysiloxanes, and combinations thereof.

Suitable synthetic polymers for use according to the teachings of the present invention can also include biosynthetic polymers based on sequences found in collagen, elastin, thrombin, fibronectin, starches, poly(amino acid), polypropylene fumarate), gelatin, alginate, pectin, fibrin, oxidized cellulose, chitin, chitosan, tropoelastin, hyaluronic acid, polyethylene, polyethylene terephthalate, poly(tetrafluoroethylene), polycarbonate, polypropylene and poly(vinyl alcohol), ribonucleic acids, deoxyribonucleic acids, polypeptides, proteins, polysaccharides, polynucleotides and combinations thereof.

The phrase “natural polymer” refers to polymers that are naturally occurring. Non-limiting examples of such polymers include, silk, collagen-based materials, chitosan, hyaluronic acid and alginate.

The phrase “biocompatible polymer” refers to any polymer (synthetic or natural) which when in contact with cells, tissues or body or physiological fluid of an organism does not induce adverse effects such as immunological reactions and/or rejections and the like. It will be appreciated that a biocompatible polymer can also be a biodegradable polymer.

The phrase “biodegradable polymer” refers to a synthetic or natural polymer which can be degraded (i.e., broken down) in the physiological environment such as by enzymes, microbes, or proteins. Biodegradability depends on the availability of degradation substrates (i.e., biological materials or portion thereof which are part of the polymer), the presence of biodegrading materials (e.g., microorganisms, enzymes, proteins) and the availability of oxygen (for aerobic organisms, microorganisms or portions thereof), carbon dioxide (for anaerobic organisms, microorganisms or portions thereof) and/or other nutrients. Aliphatic polyesters, poly(amino acids), polyalkylene oxalates, polyamides, polyamido esters, poly(anhydrides), poly(beta-amino esters), polycarbonates, polyethers, polyorthoesters, polyphosphazenes, and combinations thereof are considered biodegradable. More specific examples of biodegradable polymers include, but are not limited to, collagen (e.g., Collagen I or IV), fibrin, hyaluronic acid, polylactic acid (PLA), polyglycolic acid (PGA), polycaprolactone (PCL), poly(Lactide-co-Glycolide) (PLGA), polydioxanone (PDO), trimethylene carbonate (TMC), polyethyleneglycol (PEG), Collagen, PEG-DMA, alginate or alginic acid, chitosan polymers, or copolymers or mixtures thereof.

The phrase “non-biodegradable polymer” refers to a synthetic or natural polymer which is not degraded (i.e., broken down) in the physiological environment. Examples of non-biodegradable polymers include, but are not limited to, carbon, nylon, silicon, silk, polyurethanes, polycarbonates, polyacrylonitriles, polyanilines, polyvinyl carbazoles, polyvinyl chlorides, polyvinyl fluorides, polyvinyl imidazoles, polyvinyl alcohols, polystyrenes and poly(vinyl phenols), aliphatic polyesters, polyacrylates, polymethacrylates, acyl-sutostituted cellulose acetates, nonbiodegradable polyurethanes, polystyrenes, chlorosulphonated polyolefins, polyethylene oxides, polytetrafluoroethylenes, polydialkylsiloxanes, and shape-memory materials such as poly (styrene-block-butadiene), copolymers or mixtures thereof.

The phrase “biosorbable” refers to those polymers which are absorbed within the host body, either through a biodegradation process, or by simple dissolution in aqueous or other body fluids. Water soluble polymers, such as poly(ethylene oxide) are included in this class of polymers.

As described above, in some embodiments, at least one layer contains a fiber comprising a porogen. These are generally biosorbable materials, and are added and then removed to provide space for cell ingression. The use of such materials is described, for example, in Baker, et al., Biomaterials, 29 (2008), 2348-2358, this reference being incorporated by reference in its entirety.

It will be appreciated that more than one polymer may be used to fabricate the individual layers of the present invention. For example, each layer may be fabricated from multiple co-spun polymer or co-polymers. The term “co-polymer” as used herein, refers to a polymer of at least two chemically distinct monomers. Non-limiting examples of co-polymers which may be used to fabricate the scaffolds of the present invention include, PLA-PEG, PEGT-PBT, PLA-PGA, PEG-PCL and PCL-PLA. The use of copolymers or mixtures of polymers/copolymers provides a flexible means of providing the required blend of properties. In but one non-limiting example, functionalized poly(β-amino esters), which may be formed by the conjugate addition of primary or secondary amines with diacrylates, can provide a range of materials exhibiting a wide array of advantageous properties for this purpose. Such materials are described, for example, in Anderson, et al., “A Combinatorial Library of Photocrosslinkable and Degradable Materials,” Adv. Materials, vol. 18 (19), 2006, this reference being incorporated by reference in its entirety.

Additionally, individual polymers or co-polymers may be physically mixed and co-spun through the same spinneret.

Similarly, according to this invention, the scaffold may be comprised of a mixture of simultaneously or sequentially delivered polymers and/or copolymers. This includes mixtures of at least two natural, synthetic, biocompatible, biodegradable, non-biodegradable, and/or biosorbable polymers and co-polymers.

Other embodiments of this invention provide that at least one fiber is biodegradable in a physiological fluid, said fluids including water, saline, simulated body fluid, or synovial fluid. Further, where the scaffold comprises two or more biodegradable fibers, each can have a different biodegradation and/or biosorption profile. In certain embodiments, the biodegradation and/or biosorption profile of the at least one biodegradable fiber is chosen to approximately coincide with the rate of ingression of tissue growth. In this way, the degradation in modulus of the scaffold can be made to match or partially offset the temporal stiffening associated with ingression of the growing tissue, thus allowing a system to be designed with approximately constant temporal performance parameters.

Still other embodiments provide that the polymers, co-polymers, or blends thereof may be photolytically active, such that once electrospun, the fibers may be made to crosslink on exposure to light, thereby improving the tensile characteristics of the scaffold, and increasing the diversity and range of properties available. Non-limiting examples of such fiber materials are described in, Tan, et al., J. Biomed Matl. Res., vol. 87 (4), 2008, pp. 1034-1043, which is incorporated by reference in its entirety.

In addition to the fibers, the scaffold can also comprise a variety of additional materials, added before (e.g., during formation of the fiber), during (e.g., between individual layers) or after the formation of the scaffold laminate.

In one such case, certain embodiments provide that the scaffold comprises at least one gel. Such materials are used to improve the structural integrity of the scaffold and/or to act as a delivery system for other contained elements. Hydrogels are preferred and can comprise agarose, alginate, RGD-modified alginate, chitosan, collagen, fibrin, gelatin, hyaluronic acid, matrigel, oligo(poly(ethylene glycol)fumarate), poly(ε-caprolactone), poly(ethylene glycol), poly(glycolic acid), poly(glycolic-lactic acid), poly(lactic acid) or puramatrix.

Other embodiments describe these additional materials as comprising biofactors, therapeutic agents, particles, or cells. The materials agents can be applied to at least a portion of the scaffold, using techniques well known in the art, by coating or impregnating or at least a portion of the polymer fibers prior to or during the process of electrospinning, by co-applying them with the fibers, or by impregnating the electrospun scaffold by soaking the scaffold after spinning Such attachments can be performed using e.g., cross-linking (chemical or light mediated) of the agent with the polymer solution or the electrospun fiber formed therefrom (e.g., PLC and the agent). Additionally or alternatively, the agent can be embedded in electrospun nanofibers having the core-shell structure essentially as described in Sun et al. (e.g., see Sun et al., “Compound Core/Shell Polymer Nanofibers by Co-Electrospinning”, Advanced Materials, 15, 22:1929-1936, 2003, which is incorporated by reference for this purpose), or adhered to the fibers either directly using biocompatible adhesives or through the use of biocompatible carriers, including microsphere encapsulants.

Encapsulation techniques are generally classified as microencapsulation, involving small spherical vehicles and macroencapsulation, involving larger flat-sheet and hollow-fiber membranes. Methods of preparing microcapsules are known in the electrospinning art.

The invention further provides that these biofactors, therapeutic agents, particles, or other materials incorporated within the scaffold, as required or desired, may be biodegraded, dissolved, and/or released according to a predetermined time profile. In other embodiments, these changes occur so as to complement the entry and incorporation of cells and/or tissues within the scaffold.

The invention is flexible in allowing these biofactors, therapeutic agents, particles, or cells also to be present independently in the scaffold as at least one gradient within the composition, and this at least one gradient can either across the various dimensions of the scaffold. These gradients can be continuous or step-wise, again, as determined by the processing parameters.

In one set of embodiments, these additional materials comprise at least one therapeutic compound or agent, capable of modifying cellular activity. Similarly, agents that act to increase cell attachment, cell spreading, cell proliferation, cell differentiation and/or cell migration in the scaffold may also be incorporated into the scaffold. Such agents can be biological agents such as an amino acid, peptides, polypeptides, proteins, DNA, RNA, lipids and/or proteoglycans.

These agents may also include growth factors [e.g., a epidermal growth factor, a transforming growth factor-α, a basic fibroblast growth factor, a fibroblast growth factor-acidic, a bone morphogenic protein, a fibroblast growth factor-basic, erythropoietin, thrombopoietin, hepatocyte growth factor, insulin-like growth factor-I, insulin-like growth factor-II, Interferon-β, platelet-derived growth factor, a nerve growth factor, a transforming growth factor, a tumor necrosis factor, Vascular Endothelial Growth Factor, an angiopeptin, or a homolog or combination thereof], cytokines [e.g., M-CSF, IL-lbeta, IL-8, beta-thromboglobulin, EMAP-II, G-CSF and IL-IO, or a homolog or combination thereof], proteases [pepsin, low specificity chymotrypsin, high specificity chymotrypsin, trypsin, carboxypeptidases, aminopeptidases, proline-endopeptidase, Staphylococcus aureus V8 protease, Proteinase K (PK), aspartic protease, serine proteases, metalloproteases, ADAMTS 17, tryptase-gamma, and matriptase-2, or a homolog or combination thereof] and protease substrates.

Suitable proteins which can be used along with the present invention include, but are not limited to, extracellular matrix proteins [e.g., fibrinogen, collagen, fibronectin, vimentin, microtubule-associated protein ID, Neurite outgrowth factor (NOF), bacterial cellulose (BC), laminin and gelatin], cell adhesion proteins [e.g., integrin, proteoglycan, glycosaminoglycan, laminin, intercellular adhesion molecule (ICAM) 1, N-CAM, cadherin, tenascin, gicerin, RGD peptide and nerve injury induced protein 2 (ninjurin2)].

Additionally and/or alternatively, the scaffolds of the present invention may comprise an antiproliferative agent (e.g., rapamycin, paclitaxel, tranilast, Atorvastatin and trapidil), an immunosuppressant drug (e.g., sirolimus, tacrolimus and Cyclosporine) and/or a non-thrombogenic or anti-adhesive substance (e.g., tissue plasminogen activator, reteplase, TNK-tPA, glycoprotein IIb/IIIa inhibitors, clopidogrel, aspirin, heparin and low molecular weight heparins such as enoxiparin and dalteparin).

Examples of immunosuppressive agents which can be used to minimize immunosuppression include, but are not limited to, methotrexate, cyclophosphamide, cyclosporine, cyclosporin A, chloroquine, hydroxychloroquine, sulfasalazine (sulphasalazopyrine), gold salts, D-penicillamine, leflunomide, azathioprine, anakinra, infliximab (REMICADE), etanercept, TNF-α, blockers, a biological agent that targets an inflammatory cytokine, IL-1 receptor antagonists, and Non-Steroidal Anti-Inflammatory Drug (NSAIDs). Examples of NSAIDs include, but are not limited to acetyl salicylic acid, choline magnesium salicylate, diflunisal, magnesium salicylate, salsalate, sodium salicylate, diclofenac, etodolac, fenoprofen, flurbiprofen, indomethacin, ketoprofen, ketorolac, meclofenamate, naproxen, nabumetone, phenylbutazone, piroxicam, sulindac, tolmetin, acetaminophen, ibuprofen, Cox-2 inhibitors and tramadol.

Cytokines useful in the present invention include, but are not limited to, cardiotrophin, stromal cell derived factor, macrophage derived chemokine (MDC), melanoma growth stimulatory activity (MGSA), macrophage inflammatory proteins 1 alpha (MOP-1 alpha, 2, 3 alpha, 3 beta, 4, and 5, IL-, 11-2, IL-3, IL-4, IL-5, IL-6, IL-7, IL-8, IL-9, IL-10, IL-11, IL-12, IL-13, TNF-α, and TNF-β. Immunoglobulins useful in the present invention include but are not limited to, IgG, IgA, IgM, IgD, IgE, and mixtures thereof. Some preferred growth factors include VEGF (vascular endothelial growth factor), NGFs (nerve growth factors), PFGF-AA, PDGF-BB, PDGF-AB, FGFb, FGFa, and BGF.

Additionally, the scaffolds of the present invention can include organic and/or inorganic particles as well as the electrospun polymers. It will be appreciated that, the scaffolds of the present invention may comprise a single type of particles or alternatively may comprise two or more types of particles. As used herein, the term “particles” refers to any finely divided solid non-cellular matter, including powders, filings, crystals, beads and the like, which are capable of being integrated into a scaffold, but without interfering with the scaffolds capability to support cells.

According to one aspect of the present invention, the particles are dispensed concomitantly with the dispensing of the electrospun polymers, although from a separate dispenser e.g. by air pressure from a pneumatic activator. It will be appreciated that the concomitant dispensing of the particles from a separate dispenser can result in particles being situated between the polymeric fibers and not necessarily embedded within the fibers.

Another embodiment provides that the scaffold further comprises at least one population of cells. These populations of cells can exist as homogeneous or heterogeneous mixtures within or across at least one layer or at the interface of two individual layers, or provide a scaffold having at least one gradient across the various dimensions of the scaffold. These gradients can be continuous or step-wise, as with the other components, as determined by the processing parameters.

Techniques for seeding cells onto or into a scaffold are well known in the art, and include, without being limited to, static seeding, filtration seeding and centrifugation seeding. See, e.g., Baker and Mauck, “The effect of nanofiber alignment on the maturation of engineered meniscus constructs,” Biomaterials, 28 (2007) 1967-1977, which is incorporated in its entirety by reference for this purpose. Static seeding includes incubation of a cell-medium suspension in the presence of the scaffold under static conditions and results in non-uniformity cell distribution (depending on the volume of the cell suspension); filtration seeding results in a more uniform cell distribution; and centrifugation seeding is an efficient and brief seeding method (see for example EP19980203774).

The cells may be seeded directly onto the scaffold, or alternatively, the cells may be mixed with a gel, preferably a hydrogel, which is then absorbed onto the interior and exterior surfaces of the scaffold and which may fill some of the pores of the scaffold. Capillary forces will retain the gel on the scaffold before hardening, or the gel may be allowed to harden on the scaffold to become more self-supporting. Alternatively, the cells may be combined with a cell support substrate in the form of a gel optionally including extracellular matrix components. Certain preferred gels have been described above.

The cells which can be used according to the teachings of the present invention may comprise non-autologous cells or non-autologous cells (e.g. allogeneic cells or xenogeneic cells), such as from human cadavers, human donors or xenogeneic (e.g. porcine or bovine) donors.

The cells may comprise a heterogeneous population of cells or alternatively the cells may comprise a homogeneous population of cells. Such cells can be for example, stem cells (such as adipose derived stem cells, embryonic stem cells, bone marrow stem cells, cord blood cells, mesenchymal stem cells, adult tissue stem cells, induced pluripotential stem cells,), progenitor cells (e.g. progenitor bone cells), or differentiated cells such as chondrocytes, meniscal fibrochondrocytes, osteoblasts, osteoclasts, osteocytes, connective tissue cells (e.g., fibrocytes, fibroblasts, tenocytes, and adipose cells), endothelial and epithelial cells, or mixtures thereof. Stem cells, and especially mesenchymal stem cells are preferred.

As used herein, the phrase “stem cell” refers to cells which are capable of differentiating into other cell types having a particular, specialized function (i.e., “fully differentiated” cells) or remaining in an undifferentiated state hereinafter “pluripotent stem cells”.

Furthermore, such cells may be of autologous origin or non-autologous origin, such as postpartum-derived cells (as described in U.S. application Ser. Nos. 10/887,012 and 10/887,446). Typically the cells are selected according to the tissue being generated.

In other embodiments, these cells develop into tissue, such that the scaffolds comprise growing tissue corresponding to the cells used.

The invention also describes embodiments directed toward making the scaffolds heretofore described. Particular embodiments include a method of making an implant scaffold comprising contacting at least two layers, each layer comprising at least one fiber aligned along a major axis of said layer, the layers being positioned such that the major axis of a first layer forms an oblique angle with respect to the major axis of a second layer, said oblique angle defining a long axis within the arc of the oblique angle.

In forming the scaffold from individual layers, several methodologies are contemplated by this invention. The descriptions which follow should not be considered limiting, rather exemplary. As those skilled in the art will appreciate, numerous modifications and variations of the present invention are possible in light of these teachings, and all such are contemplated hereby

In one embodiment, each layer is individually formed and physically isolated, either by electrospinning or any other method which provides for layers of substantially aligned fibers as described herein. These physically isolated layers are then apositioned such that the major axis of layer is oriented to be consistent with the descriptions provided above for the scaffolds. In other embodiments, an individual layer or combined layers are physically isolated and additional layers are applied using direct electrospinning onto the layer or layers. In one non-limiting example, an initially electrospun layer is removed from the mandrel, rotated by the desired oblique angle, re-mounted onto the mandrel, and a second layer of fiber is applied. A skilled artisan will appreciate that any combination of builds may be used, provided the final product is consistent with the described scaffolds.

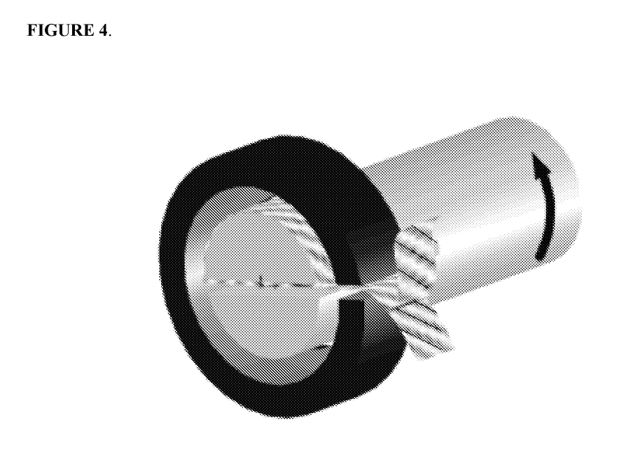

To form more complicated anatomical constructs using these layered scaffolds, one embodiment provides that two or more layers are wound around a cylindrical form, such that the long axis is made to be circumferential to the center-line axis of the cylindrical mold. FIG. 4. Other embodiments provide for non-cylindrical molds. Similarly, conformance of layered scaffold laminates to non-planar molds, or around non-planar objects may be used to provide desired constructs.

Similarly, additional embodiments provide that the individual layers be joined, either physically or chemically. Such joining may comprise chemical crosslinking, including but not limited to photocatalytic crosslinking In the latter embodiment, the prior inclusion of at least one photolytically active fiber in adjacent layers is preferred. Other embodiments provide that said joining comprises applying adhesive to fibers of one or both of two adjacent layers. Such adhesive may be activated thermally, chemically, photolytically, or by microwave radiation. Other embodiments provide that the fibers of two adjacent layers may respond to weld together by the general or local application of heat or pressure.

In still other embodiments, adjacent layers may be joined by growing tissue within the scaffold or by the application and thickening of gel materials.

In still other embodiments, where at least one layer contains a porogenic material, that material may be removed before the application of at least one population of cells, and/or the application of at least one therapeutic agent, biofactor, catalyst, or mixture or combination thereof. The methods used to provide for these latter materials are described above, and are known to those skilled in the art to which this invention pertains.

Still other embodiments provide that tissue be grown on or within the scaffold. This may be done in vitro, in vivo, or in a process combining the two methods.

This invention also provides for one or more kits containing a packaged sterilized implant scaffold, as described herein. These kits comprise any of the various embodiments, including one or more of the properties and characteristics described herein, and may include at least one plate for connecting to and/or distributing the forces of the neighboring bone over the surface of the implant. For example, certain embodiments provide that the packaged kit includes a plate for supporting the upper vertebra, another plate supporting the lower vertebra, as well as the scaffolding material. Kits which include at least one shaped supporting plate are also within the scope of the present invention.

In other embodiments, the kits may include a carrier for the scaffold. Other embodiments provide that the scaffold is provided in a sterilized package with an insertion adapter, such as a head, holder, or other carrier. The insertion adapter may be configured to retain the scaffold and to engage an insertion tool body.

In still other embodiments, the kits provide a jacket surrounding the scaffold, the jacket constraining hydration of the scaffold to the partial or fully hydrated state.

The invention further provides for embodiments describing the use of these implant scaffolds in patients. One such embodiment provides a method of treating a mammalian patient comprising: (a) assessing the need to repair or replace at least one body part of said patient; (b) deciding that implanting a scaffold to facilitate the repair or replacement of said body part is a viable treatment for said patient; and (c) implanting into said patient an implant scaffold comprising at least two overlapping layers, each layer comprising at least one fiber aligned along a major axis of said layer, the layers being positioned such that the major axis of a first layer forms an oblique angle with respect to the major axis of a second layer, said oblique angle defining a long axis within the arc of the oblique angle. The invention further provides that these substantially anatomically shaped solids can be used as scaffold implants, to replace or repair their corresponding anatomical part. Such scaffolds may be used, for example, to replace or repair AF, knee menisci, connective tissue, or bone.

For medical applications, the anatomically shaped scaffolds can be implanted to treat diseases characterized by connective tissue or meniscal damage or loss. As used herein, the phrase “connective tissue” refers to tissues which surround, protect, bind and support all of the structures in the body. Examples of connective tissues include, but are not limited to, cartilage (including, elastic, hyaline, and fibrocartilage), collagen, adipose tissue, reticular connective tissue, embryonic connective tissues (including mesenchymal connective tissue and mucous connective tissue), tendons, ligaments, and bone.

Additionally, the unique consequence of the internal organization of the present scaffolds makes them particularly useful for implants to repair or regenerate blood vessels, corneas, rotator cuff tendons, urinary bladder walls, diaphragms, and other biologic orthopedic or cardiovascular laminates which would benefit from the angle-ply structures of the present invention.

Patients for which such implants may be considered include mammals, said mammals including humans. It should be appreciated that while not necessarily required in all applications, it is at least highly preferred that the materials of construction appropriate regulatory approval, at least for use in human patients; e.g., in the United States, approval by the U.S. Food and Drug Administration. Other countries have similar approval requirements.

As used herein, the term “treating” refers to inhibiting or arresting the development of a disease, disorder or condition and/or causing the reduction, remission, or regression of a disease, disorder or condition in an individual suffering from, or diagnosed with, the disease, disorder or condition, or repairing breaks, rips, or tears in the tissue, such as a complete or partial replacement of an intervertebral disc. Those of skill in the art will be aware of various methodologies and assays which can be used to assess the development of a disease, disorder or condition, and similarly, various methodologies and assays which can be used to assess the reduction, remission or regression of a disease, disorder or condition.

Those skilled in the art are capable of determining when and how to pre-treat (for example, including appropriate sterilization methods) and implant the scaffold to thereby induce tissue regeneration and treat the pathology. Embodiments of the present invention include such sterilization methods when taken in connection with the use or manufacture of the scaffolds of the claimed invention. The site of implantation is dependent on the disease to be treated. For example, if the pathology to be treated is a torn meniscus the scaffold is seeded with chondrocytes or stem cells and following the required days in culture the scaffold is preferably implanted in the damaged knee. Similarly, if the pathology to be treated is torn or degraded intervertebral discs, the scaffold is seeded with AF and nucleus pulpous cells or stem cells and following the required days in culture, the scaffold is preferably implanted in the spine. While knee menisci and intervertebral discs are described here, the invention is also applicable and useful for implantation into other joints, and for treatment of and attached to bone, muscle, or tendon.

The scaffolds of the present invention are suitable for ex vivo tissue formation to be utilized in surgical procedures. According to another embodiment, tissue formation is effected in vivo—in this case the solid scaffold supported cells are typically implanted into the subject immediately following seeding.

Embodiments in which the substance comprises cells include cells that can be cultured in vitro, derived from a natural source, genetically engineered, or produced by any other means. Any natural source of prokaryotic or eukaryotic cells may be used. Embodiments in which the matrix is implanted in an organism can use cells from the recipient, cells from a conspecific donor or a donor from a different species, or bacteria or microbial cells. Cells harvested from a source and cultured prior to use are included.

As those skilled in the art will appreciate, numerous modifications and variations of the present invention are possible in light of these teachings, and all such are contemplated hereby.

### EXAMPLES

### Example 1

### Nanofibrous Scaffold Fabrication

Aligned nanofibrous scaffolds were generated via electrospinning Briefly, poly(ε-caprolactone) (PCL) were dissolved at 143 mg/mL in equal parts tetrahydrofuran and N,N-dimethylformamide, then extruded at 2.5 mL/h through a spinneret charged to $13 kV. The resulting nanofibrous jet was collected on a grounded mandrel rotating at I0 m/s and located 20 cm from the spinneret. Aluminum shields on either side of the spinneret were charged to +9 kV to focus the jet. The spinneret was fanned back and forth to ensure uniform fiber deposition. Mats of ca. 250 μm thickness were electrospun to match the natural lamellar thickness of the annulus fibrosus.

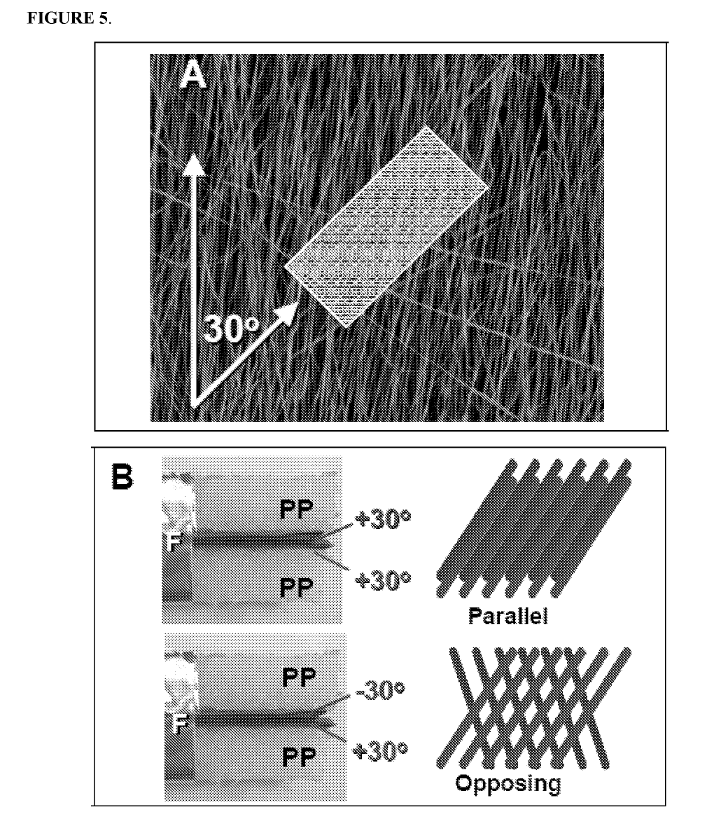

Rectangular scaffolds (5 mm×30 mm) were excised from the nanofibrous mat with their long axis rotated 30° from the prevailing fiber direction. This produced aligned scaffolds whose fiber angle (FIG. 5A) reflected the oblique alignment of collagen fibers within a single lamella of the annulus fibrosus.

### Example 2

### Isolation and Seeding of MSCs on Nanofibrous Scaffolds

The single lamellar scaffolds of Example 1 were seeded with bovine MSCs and cultured in vitro in a media formulation supportive of fibrocartilaginous differentiation. MSCs were isolated from femoral and tibial bone marrow of 3-6 month old calves and expanded to passage 2 as described in B. M. Baker and R. L. Mauck, Biomaterials 28 (11), 1967 (2007). Scaffolds were hydrated by sequential washes in 100%, 70%, 50% and 30% ethanol and finally Phosphate Buffered Saline (PBS). Before seeding, scaffolds were incubated overnight in 20 μg/mL fibronectin. 50 μL of cell solution (1×10⁷ cells/mL) were applied to one side, followed by incubation at 37° C. for one hour. Scaffolds were then turned and an additional 50 μL of cell solution applied to the other side. After two hours further incubation, samples were transferred to chemically defined media (DMEM [Dulbecco's Modified Eagle Medium], 0.1 μM dexamethasone, 40 μg/mL LProline, 100 μg/mL Sodium Pyruvate, 1% Insulin, Transferrin, Selenium/Premix, and 1% penicillin, streptomycin and fungizone supplemented with 10 ng/mL Transforming Growth Factor β3). Media was replaced twice weekly for the duration of the study.

### Example 3

### Formation of Laminated Scaffolds

After two weeks, the lamellae of Example 2 were brought into apposition between pieces of porous polypropylene and wrapped with a foil sleeve (0 weeks, FIG. 5B). Bilayers were formed with the nanofibers in adjacent lamellae running either parallel at +30° (Parallel) or in opposing directions of +30° and −30°. After two additional weeks of culture, the external supports were removed and laminates remained intact.

### Example 4

### Biochemical Analyses

For biochemical analyses, samples were digested for 16 hours in papain at 60° C., then analyzed for s-GAG content using the 1,9-dimethylmethylene blue dye-binding assay, for orthohydroxyproline (OHP) content (after acid hydrolysis) to determine collagen content by reaction with chloramine T and dimethylaminobenzaldehyde, and for DNA content using the PicoGreen dsDNA Quantification kit. OHP content was converted to collagen with a ratio of 1:7.14 (OHP:collagen).

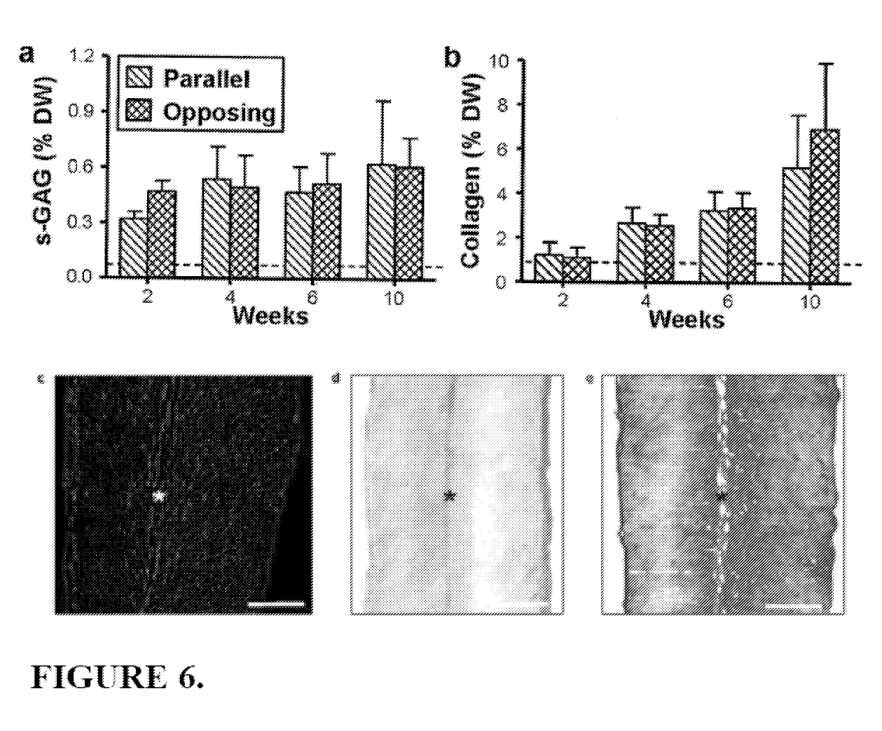

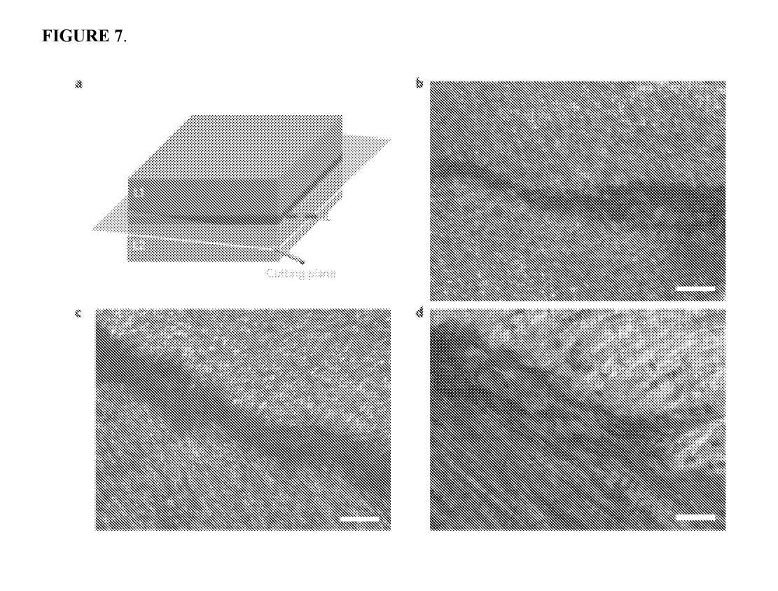

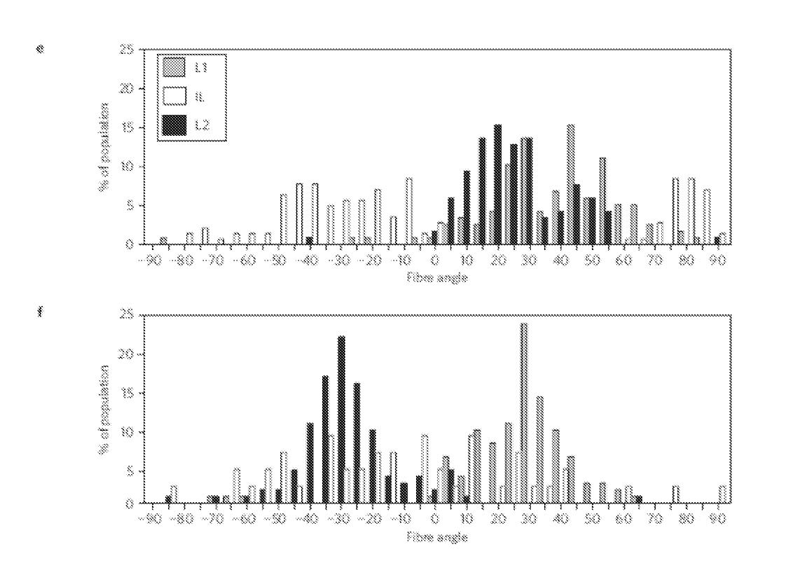

Biochemical analyses through 10 weeks of in vitro culture revealed significant accumulation of sulphated glycosaminoglycans (FIGS. 6A, 7D) and collagen (FIGS. 6B, 7E), two of the primary extracellular components of the annulus fibrosus, within both Parallel and Opposing bilayers. In the two weeks preceding bilayer formation, cells began infiltrating through the thickness with a dense cell layer at the scaffold periphery (not shown). After bilayer formation, these outer cell layers fused in the contacting region between two lamellae, forming a thin inter-lamellar space that became more pronounced with culture duration (asterisk, FIGS. 6C to 6E). Glycosaminoglycans and collagen were distributed throughout each lamella, and within this inter-lamellar space, indicating the successful formation of a biologic interface between the two layers (FIGS. 6C, 6D). MSCs infiltrated into the scaffold, but remained most densely populated at the surfaces (FIGS. 6E). No differences in cell, glycosaminoglycan or collagen quantity and localization were observed between Parallel and Opposing bilayers, nor were any differences observed compared to single lamella constructs maintained under identical culture conditions (normalized to dry weight).

### Example 5

### Histology

Samples (n=3) were cryo-sectioned as described in N. L. Nerurkar, et al., Spine 33 (25), 2691 (2008). Paraformaldehyde-fixed sections were stained for cell nuclei (40,6-diamidino-2-phenylindole, DAPI), glycosaminoglycans (Alcian Blue) and collagen (Picrosirius Red). DAPI-stained sections were visualized at 20× on a Nikon T30 inverted fluorescent microscope. Alcian Blue and Picrosirius Red stains were visualized on an upright Leica DMLP microscope. Annulus fibrosus from skeletally mature bovine caudal discs were processed identically. Quantitative polarized light microscopy was performed on Picrosirius Red stained sections to quantify collagen alignment as described previously 45. Briefly, grayscale images were collected (20×) at 10° increments using a green bandpass filter (BP 546 nm) with crossed analyzer and polarizer coordinately rotated through 90″. This was repeated with the filter replaced by a λ compensator. Custom software was then used to determine collagen fiber orientations for a series of nodes within the central portion of each region of interest.

Parallel and Opposing bilayers were visualized obliquely to observe collagen alignment simultaneously across layers (FIG. 7A). When stained for collagen and viewed by polarized light microscopy, intra-lamellar collagen was highly birefringent in both Parallel (FIG. 7B) and Opposing (FIG. 7C) bilayers, indicating that collagen was aligned with the underlying nanofibrous scaffold. Collagen deposited into the inter-lamellar region, however, was disorganized. As indicated by the hue of birefringence, intralamellar collagen was co-aligned within Parallel bilayers and oriented along two opposing directions for Opposing bilayers. Additionally, collagen organization within Opposing bilayers compared favorably with similarly prepared sections of annulus fibrosus (FIG. 7D), indicating successful replication of the multi-scale collagen architecture of the native tissue. Quantitative polarized light analysis confirmed co-alignment of intra-lamellar collagen in Parallel bilayers, indicated by overlapping fiber populations (FIG. 7E) at approximately +30° from the long axis. However, fiber populations within Opposing bilayers demonstrated two distinct peaks in orientation: +30° and −30° from the long axis. For both Parallel and Opposing bilayers, inter-lamellar matrix orientations were widely distributed with no single distinct peak, confirming lack of alignment in this region. Opposing bilayers present the first instance in which an engineered tissue has successfully replicated the angle-ply laminate architecture of the annulus fibrosus.

### Example 6

### Uniaxial Tensile Testing and Biochemical Analyses

After measuring cross-sectional area using a custom laser device, samples (n=5) were clamped with serrated grips and loaded into an Instron 5542 testing device. Gauge length was determined as the distance between grips. All testing was performed in a PBS bath. The mechanical testing protocol consisted of: (1) a nominal tare load of 0.1 N applied at 0.1% strain/sec, followed by stress relaxation for 5 minutes, (2) 15 preconditioning cycles to 0.1% strain at 0.05% strain/sec, and (3) a quasi-static elongation at 0.1% strain/sec until failure. Strain was determined as extension normalized to gauge length; stress was computed as the load normalized to initial cross-sectional area. Modulus was computed as the slope of the stress-strain plot, determined by regression to the linear portion of the curve.

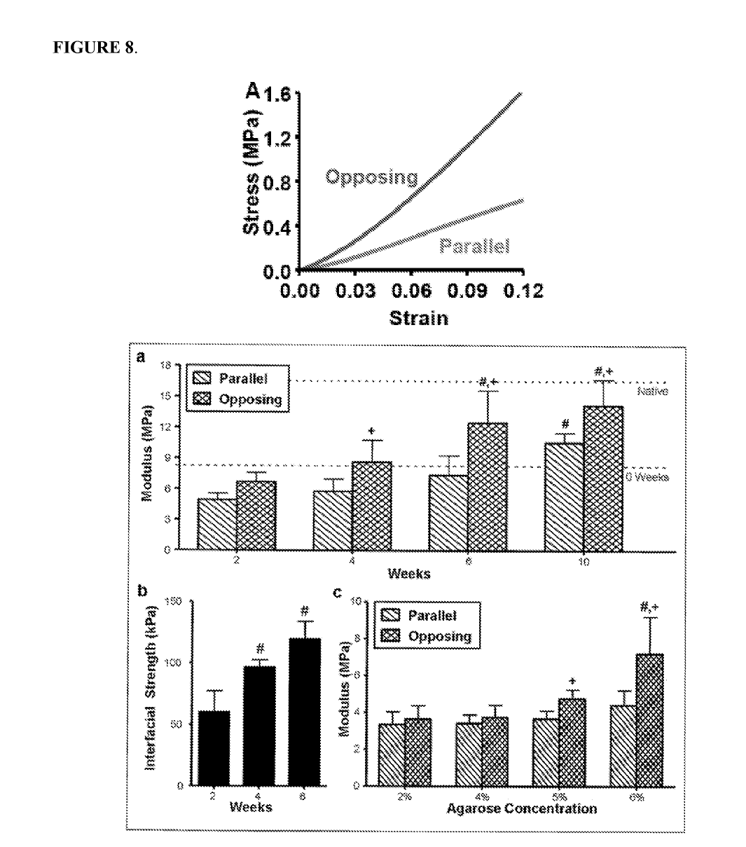

For both Parallel and Opposing bilayers, tensile moduli increased with culture duration when compared to single lamellar moduli at the time of bilayer formation (0 weeks, FIG. 8A). In the first few weeks after apposition, a reduction in modulus was observed due to swelling; stiffness steadily increased for both groups for all time points (not shown). Interestingly, Opposing bilayer moduli were significantly greater than Parallel bilayers by as early as 4 weeks, and remained higher through completion of the study at 10 weeks (p≦0.05). In fact, Opposing (14.2±2.5 MPa)—but not Parallel (10.6±0.9 MPa)—bilayers achieved a tensile modulus by 10 weeks that approximates the circumferential tensile modulus of the annulus fibrosus to within 15% (17.3 MPa). This is, to date, the closest an engineered tissue has come to matching the tensile properties of the annulus fibrosus.

### Example 7

### Bilayer Lap Testing

Nanofibrous scaffolds of approximately 1 mm thickness were prepared as above and excised along the fiber direction. Two weeks of after seeding with MSCs, samples (n=5) were placed in apposition with a 20 mm overlap and secured with porous polypropylene and foil and cultured as above. Lap tests were performed by gripping the overhang on either end of the bilayer and extending to failure at 0.2 mm/s. Interface strength was determined from the maximum force normalized to overlap area.

In order to isolate the functional properties of the interface, intra-lamellar deformations were reduced by: (1) using thicker (1 mm) scaffolds and (2) aligning nanofibers parallel to the loading axis in both lamellae. Scaffolds of this size and orientation were two orders of magnitude stiffer than the interface, allowing direct measurement of interfacial properties with minimal intra-lamellar deformation. Indeed, failure occurred consistently at the interface. Interfacial strength increased by nearly 3-fold within the first six weeks after bilayer formation (FIG. 8B). This suggests that while after 2 weeks of in vitro culture, load can be transmitted across the inter-lamellar space, the strength of bonding continues to increase with culture duration. This supports the potential role for lamellar bonding in explaining the functional disparity between Parallel and Opposing bilayers.

### Example 8

### Acellular Bilayers

Aligned nanofibrous scaffolds of approximately 1 mm thickness were excised at 30° from the fiber direction. Agarose was dissolved in PBS at 2, 4, 5, and 6% w/v and melted by autoclaving. Molten agarose was applied between layers of scaffold, and allowed to set at room temperature. No significant difference was observed in cross-sectional area across all concentrations, indicating controlled, reproducible interface formation. Resulting bilayers (n=5) were tested in uniaxial tension as described above.

Opposing bilayer modulus increased with increasing agarose concentration (p≦0.05) and hence interlamellar bonding strength, while Parallel bilayers were unchanged (FIG. 8C). It is notable that while the modulus of agarose alone increases only on the order of a 0.1 MPa with increasing concentration from 2% to 6%, the Opposing bilayer modulus increased by 3.5 MPa.

### Example 9

### Statistics

Significance for the data in Examples 3 through 8 was established by p≦0.05 as determined by two-way ANOVA with a Tukey's post hoc test for independent variables of bilayer orientation (Parallel/Opposing) and culture duration (or agarose concentration). All data are reported as mean±standard deviation. A complete biologic replicate was completed for all experiments, confirming the obtained results.

### Example 10

### Scaffold Fabrication

Aligned poly(ε-caprolactone) (PCL) fiber mats were formed through the process of electrospinning Briefly, 8 g poly(ε-caprolactone) (PCL, Sigma Aldrich, batch # 00702CE) was dissolved at 37° C. overnight in 56 mL of equal parts tetrahydrofuran and N,N-dimethylformamide (DMF). The PCL solution was extruded at 2.5 mL/h through a spinneret charged to +13 kV, generating a nanofibrous jet that was collected onto a grounded mandrel rotating at 10 m/s to instill alignment in the depositing fibers. Aluminum shields on either side of the spinneret were charged to +9 kV to focus the jet. The spinneret was fanned back and forth to ensure uniform deposition, as described in Baker, Biomaterials, 29, 2348-2358 (2008). The fibers were collected for 3 hours to generate a mesh of approximately 250 micron thickness. Rectangular samples (3 mm×30 mm) were excised from the mesh along the fiber direction.

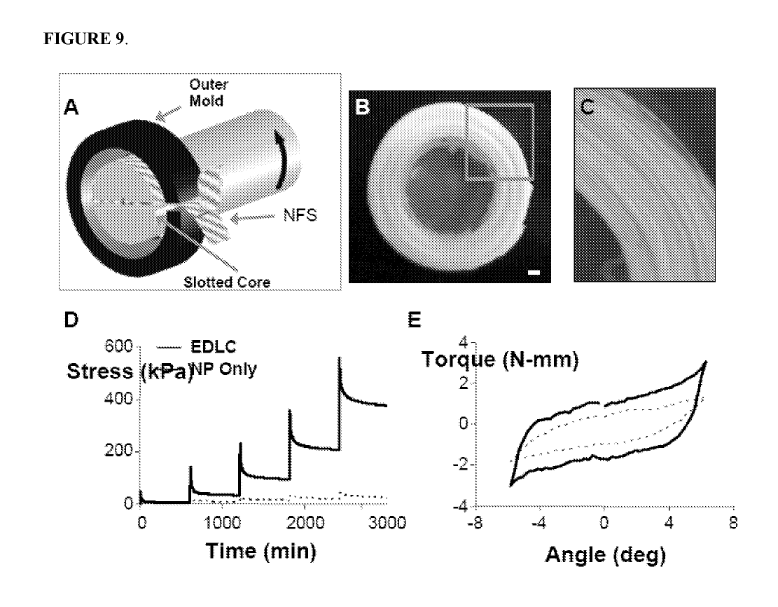

Strips (3 mm wide) were laid end-to-end with parallel fiber alignment, and spot welded to achieve strips of 150 mm final length. Strips were wrapped concentrically within a custom mold by feeding one end into a slotted core (5 mm diameter), which was then rotated (FIG. 9A) until an outer diameter of 10 cm was achieved. Rabbit disc and NP area were used to define these geometries. The core was then removed and the space filled with 5% agarose. To determine the contribution of the NP in the DAPS, a 5% agarose gel was also cast in the shape of the NP region of the DAPS.

### Example 11

### Mechanical Testing

DAPS and agarose-only NP regions of the scaffold of Example 10 were tested in compression and torsion. For compression, 25% total strain was applied in 5% increments at 1%/sec using an impermeable platen. Load was recorded as constructs relaxed to equilibrium (10 min) for each step of compressive deformation. For torsion testing, each construct was first compressed between 120 grit sandpaper-surfaced platens to 25% strain and allowed to relax for 5 min. Next, 10 cycles of ±6″ torsion were applied at 0.05 Hz using a custom-built micro-torsion device as described in Espinoza Orias A A, et al., “Rat disc torsional mechanics: effect of lumbar and caudal levels and axial compression load,” Spine J. 2009 March; 9(3): 204-209. Torque and rotation data were collected and are presented from the 10^{th} cycle.

Mechanical testing showed that the scaffold exhibited compressive stress relaxation and nonlinear torsion responses (FIGS. 9D and 9E), two traits of native disc. The compressive and shear moduli were each an order of magnitude higher for the DAPS than for the NP-only agarose region. Torsion testing demonstrated nonlinearity that is consistent with physiological disc behavior, with an increasing stiffness at angles greater than 4°. These findings confirm that the AF ring surrounding the NP confers compressive and torsional stiffness to the DAPS structure similar to the mechanical interplay between the NP and AF that occurs with native disc loading.

### Example 12

### Short-Term Culture Study: AF Cell Isolation and Analysis of Cell-Seeded Scaffolds

Outer AF tissue was excised from adult bovine caudal discs and minced before plating on tissue culture plastics in basal media (high glucose DMEM containing 1% Penicillin, Streptomycin, Fungizone and 10% Fetal Bovine Serum) as in Nerurkar N. L., et al., “ISSLS prize winner: Integrating theoretical and experimental methods for functional tissue engineering of the annulus fibrosus,” Spine 2008 Dec. 1;33(25): 2691-2701. Adherent cells were collected after two weeks and expanded to passage two in basal medium. A similar construction method to that described above was followed, now using oriented fiber strips that were excised 30° from the fiber direction. Prior to cell seeding, strips were sterilized through a graded series of ethanol washes (100%, 70%, 50%, 30%, 30 minutes per step), terminating in phosphate buffered saline (PBS), and then coated overnight in fibronectin (20 μg/mL). Constructs were seeded at 1.5×10⁶ cells per side and pre-cultured in a chemically defined growth media containing 10 ng/mL TGF-β3 for one week. The AF region of the DAPS was formed by coupling two strips with opposing orientations of +30° and wrapping as above, such that each rotation increased the lamellar number by 2, with alternating fiber directions between adjacent lamellae (FIG. 9A).

These AF-only constructs were cultured for 7 days after wrapping in chemically defined medium (above). Constructs were dehydrated and imaged by SEM (n=2) or cryotomed to 8 or 25 μm thickness (n=2). A subset of samples was sectioned oblique to the transverse axis to visualize in-plane cell and collagen orientations across lamellae interfaces. Sections were stained for cell nuclei (DAPI) and/or filamentous actin (AlexaFluor-phalloidin) or for collagen (Picrosirius Red).

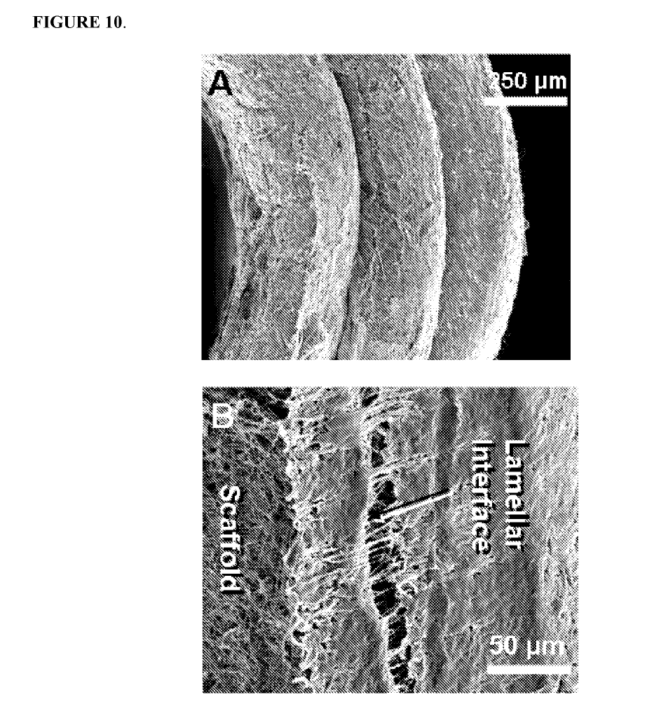

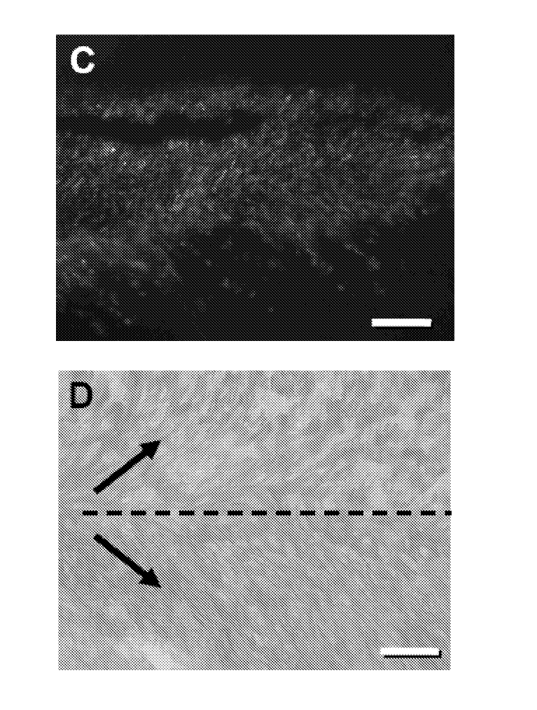

In short-term in vitro culture with bovine AF cells, AF regions successfully replicated the meso-scale multi-lamellar structure of the native tissue (FIG. 10A). Over the first week of culture, matrix deposition was evident at the lamellar interfaces when viewed under SEM (FIG. 10B). At this early time point of culture, cells colonized the interface, with limited infiltration to intra-lamellar compartments. Oblique histological sections through the interface showed actin staining (indicative of cell organization) in opposing directions in adjacent lamellae (FIG. 10C). Picrosirius Red staining of collagen likewise showed oriented ECM deposition along these alternating directions between adjacent lamellae (FIG. 10D).

Resident cells adopted an elongated morphology and were oriented in parallel within each lamella, with the direction of alignment alternating between adjacent lamellae. Both cell shape and organization closely mimic the behavior of AF precursor cells during development of the embryonic disc. Indeed, early ECM deposition was organized along the local direction of cell alignment, resulting in collagen rich lamellae that replicated the angle-ply organization of the native AF.

### Example 13

### Long-Term Study: Preparation of Scaffolds

For MSC isolation, bone marrow was isolated from femurs and tibiae of 3-6 month old calves as described above and plated on tissue culture plastic in basal medium. After initial colony formation, cells were expanded to passage 2 as described above for AF cells.

For long term analysis of DAPS maturation, strips (30° orientation) were seeded with bovine MSCs at 2 weeks and cultured in chemically defined media as above. At 0 weeks, strips were paired into bi-layers with opposing ±30° fiber orientations and wrapped concentrically as above. After 1 week to allow for stabilization of the AF region, the central lumen (5 mm diameter) was filled with MSCs encapsulated in 2% agarose at a 20×10⁶ cells/mL. Fully formed DAPS were cultured for an additional 6 weeks in 8 mL of chemically defined media supplemented per construct, with media changed twice weekly. At 1 week, when agarose encapsulated MSCs were delivered to the central lumen of DAPS, NP-only discs seeded with MSCs were also formed (5 mm diameter and 3 mm thickness). These samples were cultured identically and in parallel to determine the effect of the AF region on NP growth and maturation in the DAPS structures.

### Example 14

### Long-Term Study: Histology

At 1, 3, and 6 weeks after formation, two samples were frozen in Optimal Cutting Temperature freezing medium, cryotomed in the axial plane at 16 pm thickness, and subsequently stained with DAPI (4′,6-diamidino-2-phenylindole) for cell nuclei, Alcian Blue for proteoglycans, and Picrosirius Red for collagen. Additional samples were sectioned obliquely, stained with Picrosirius Red, and visualized via polarized light microscopy.

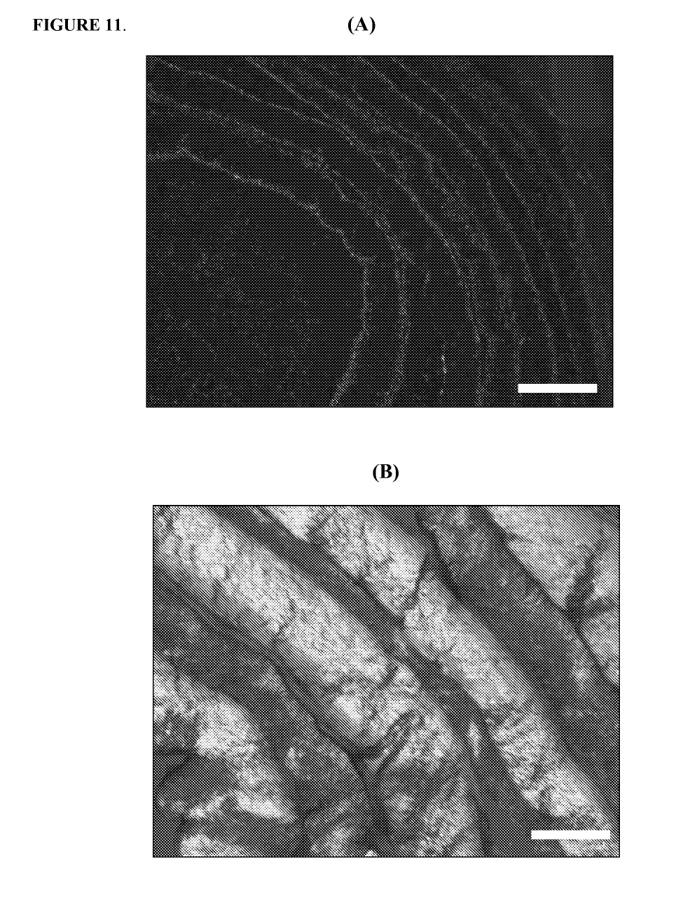

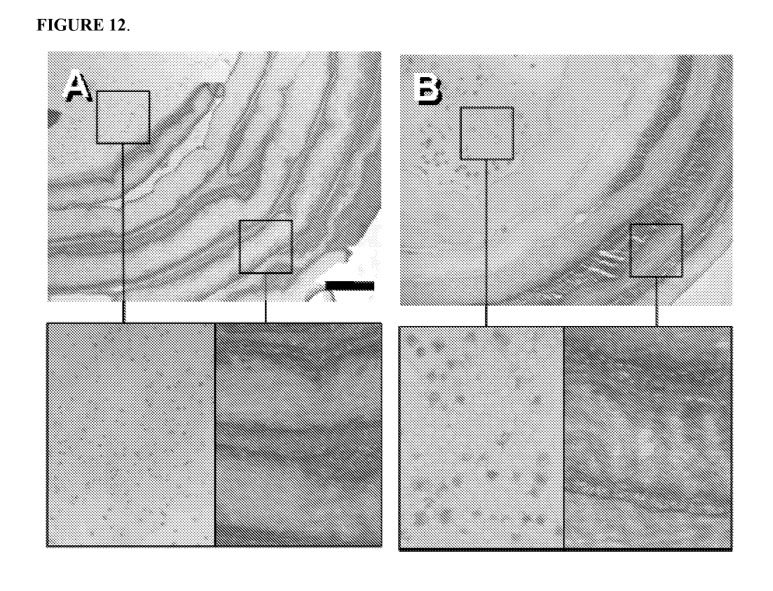

DAPI staining 1 week after formation revealed MSCs distributed homogeneously throughout the NP agarose region and at lamellar boundaries in the AF region (FIG. 11A). By 6 weeks, DAPS had matured with prominent deposition of ECM in both the NP and AF regions. Polarized light microscopy of Picrosirius Redstained oblique sections showed successful replication of native angle-ply collagen architecture, as indicated by the alternating birefringent hues in adjacent lamellae (FIG. 11B). Staining for GAG (FIG. 12A) and collagen (FIG. 12B) showed accumulation of these molecules in both regions. Notably, staining for both extracellular components appeared to decrease in intensity with progression from the outer AF to the inner AF.

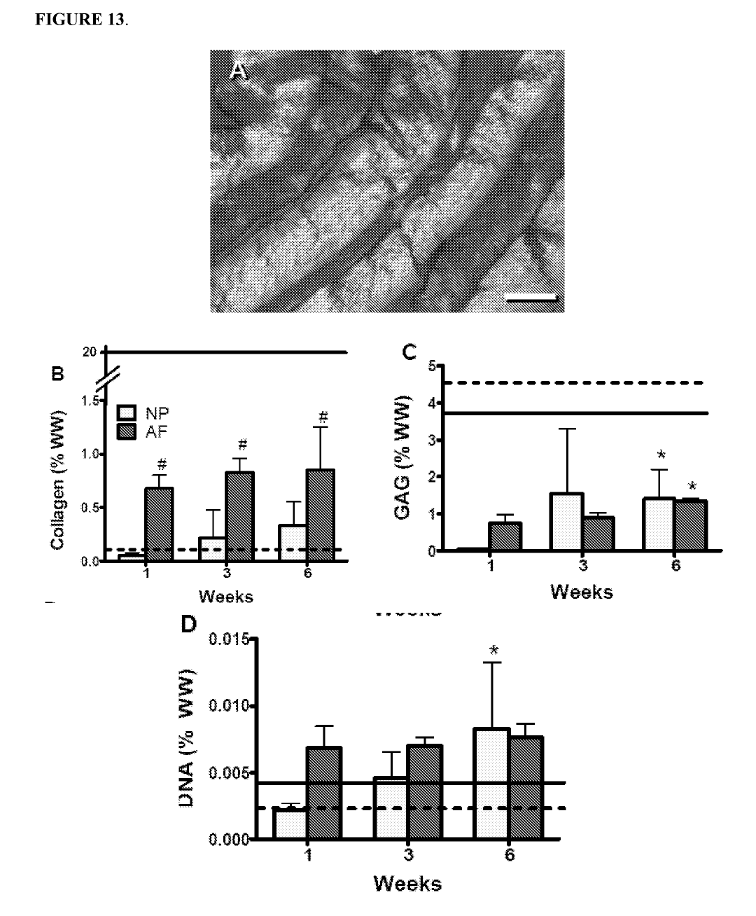

Biochemical analysis confirmed differing ECM content in the NP and AF regions. At 1 week, DNA content (per wet weight) was higher in the AF region, which had been pre-cultured for 2 weeks (FIG. 13A, p≦0.05). DNA content increased in the NP region with culture, but did not change further in the AF region. At 6 weeks, comparable DNA content was observed in both regions. Similarly, both collagen and GAG content increased in the NP region with culture duration (FIG. 13B, C). Both GAG and collagen were higher in the AF region at 1 week. At 6 weeks, GAG content in the NP and AF regions were not different from one another, but both were higher than at 1 week (p≦0.05). Conversely, collagen content remained higher in the AF region throughout the culture period. By 6 weeks, the DAPS attained DNA contents in both the NP and AF region that were similar to native disc.

### Example 15

### Long Term Study: Mechanical Testing

Briefly, the scaffolds were tested in unconfined compression between impermeable platens. Samples were pre-equilibrated for 5 minutes with a creep load of 2 grams, followed by the application of a single 10% compressive strain. Equilibrium modulus was derived from the equilibrium stress and the sample geometry, and the percent relaxation from peak to equilibrium stress. After mechanical testing, the DAPS was separated into AF and NP sub-regions, and these sub-regions assayed for collagen, GAG, and DNA content as in Mauck R L, et al., “Chondrogenic differentiation and functional maturation of bovine mesenchymal stem cells in long-term agarose culture,” Osteoarthritis Cartilage 2006 February; 14(2): 179-189. Lapine lumbar AF and NP (n=5) plugs were included in these biochemical analyses to provide native tissue concentration of these biochemical constituents.

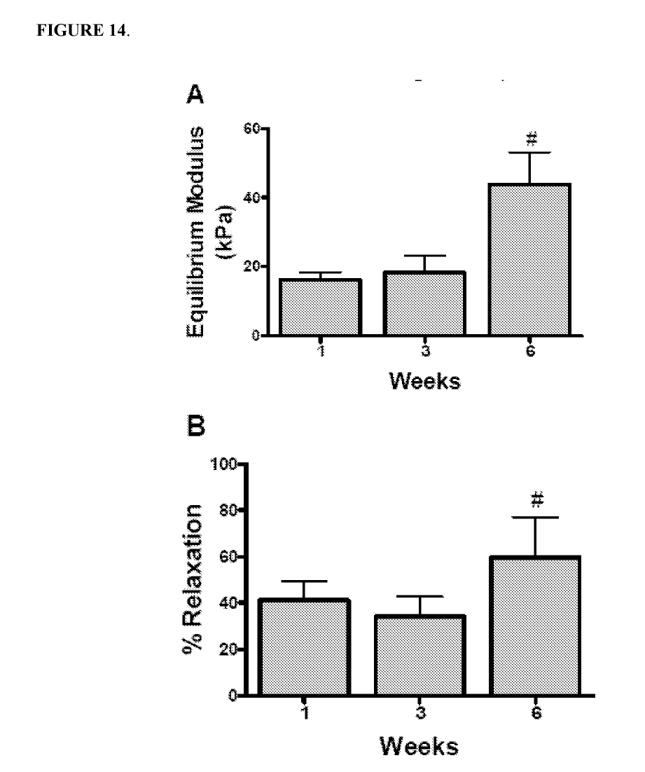

Along with ECM accumulation, the scaffolds' compressive equilibrium modulus and percent stress relaxation increased with time in culture. Specifically, the equilibrium modulus increased over two-fold to 45 kPa by 6 weeks (FIG. 14A, p≦0.05). Likewise, the % relaxation increased over this same time course (FIG. 14B, p≦0.05, from ca. 40% to ca. 60%). NP-only constructs cultured without an AF region reached 148 kPa and 72% relaxation over this time course.

### Example 16

### Statistical Analysis

Significant difference amongst quantitative outcome measures for long term scaffold cultures, as presented in Examples 11 through 15, was determined by two-way ANOVA (Analysis Of Variables) with Tukey's post hoc (p≦0.05).

What is claimed:

1. An implant scaffold comprising at least two overlapping layers, each layer comprising at least one fiber aligned along a major axis of said layer, the layers being positioned such that the major axis of a first layer forms an oblique angle with respect to the major axis of a second layer, said oblique angle defining a long axis within the arc of the oblique angle.

2. The scaffold of claim 1 wherein the long axis bisects the oblique angle.

3. The scaffold of claim 1 wherein the major axis of each of the first and second layer is independently oriented in the range of 10° to 80° with respect to the long axis.

4. The scaffold of claim 1 wherein the major axis of each of the first and second layer is independently oriented in the range of 20° to 45° with respect to the long axis.

5. The scaffold of claim 1 wherein the major axis of each of the first and second layer is independently oriented in the range of 25° to 35° with respect to the long axis.

6. The scaffold of claim 1 wherein at least one fiber is electrospun.

7. The scaffold of claim 1 wherein, where three or more layers are present, the major axis of at least one layer is parallel to the major axis of at least one other layer.

8. The scaffold of claim 1 wherein, where three or more layers are present, none of the major axes of any layer is parallel to the major axis of any other layer.

9. The scaffold of claim 1 wherein at least one layer is in the range of 50 microns to 500 microns thick.

10. The scaffold of claim 1 wherein at least one layer is in the range of 200 microns to 400 microns thick.

11. The scaffold of claim 1 where each layer is in the range of 50 nm to 500 nm thick.

12. The scaffold of claim 1 where each layer is in the range of 200 nm to 400 nm thick.

13. The scaffold of claim 1 wherein at least one fiber of at least one layer is biodegradable.

14. The scaffold of claim 1 further comprising a least one population of cells.

15. The scaffold of claim 14 wherein at least one population of cells comprises stem cells.

16. The scaffold of claim 1 further comprising growing tissue.

17. The scaffold of claim 1 further comprising at least one therapeutic agent, biofactor, catalyst, or mixture or combination thereof.

18. The scaffold of claim 1 further comprising at least one biocompatible hydrogel layer.

19. The scaffold of claim 18 where the at least one biocompatible hydrogel layer comprises agarose, alginate, RGD-modified alginate, chitosan, collagen, fibrin, gelatin, hyaluronic acid, matrigel, oligo(poly(ethylene glycol)fumarate), poly(ε-caprolactone), poly(ethylene glycol), poly(glycolic acid), poly(glycolic-lactic acid), poly(lactic acid) or puramatrix.

20. The scaffold of claim 18 where the at least one biocompatible hydrogel is compatible with stem cells.

21. The scaffold of claim 1 wherein at least one fiber from at least one layer is chemically or physically joined to at least one fiber in at least one other layer.

22. The scaffold of claim 1 wherein at least one fiber from at least one layer is capable of chemically crosslinking with at least one fiber in at least one other layer.

23. The scaffold of claim 22 wherein at least one fiber in at least one layer is chemically crosslinked to at least one fiber in at least one other layer.

24. The scaffold of claim 21 wherein at least one fiber in at least one layer is thermally or adhesively joined to at least one fiber in at least one other layer.

25. The scaffold of claim 21 wherein at least one fiber in at least one layer is joined by growing tissue to at least one fiber in at least one other layer

26. The scaffold of claim 1 wherein the modulus, when measured along the long axis, is greater than the modulus of any individual layer, when measured along the same directional axis.

27. The scaffold of claim 26 wherein the modulus, when measured along the long axis, is at least 30% greater than the modulus of any individual layer, when measured along the same directional axis.

28. The scaffold of claim 26 wherein the modulus, when measured along the long axis, is at least 50% greater than the modulus of any individual layer, when measured along the same directional axis.

29. The scaffold of claim 1 wherein the modulus, when measured along the long axis, is at least 6 MPa.

30. The scaffold of claim 1 wherein the modulus, when measured along the long axis, is at least 16 MPa.

31. The scaffold of claim 1 wherein the long axis is circumferential to a center-line axis.

32. The scaffold of claim 31 wherein the composition and/or fiber orientation of the scaffold varies with the radial distance from the center-line axis.

33. A method of making an implant scaffold comprising contacting at least two overlapping layers, each layer comprising at least one fiber aligned along a major axis of said layer, the layers being positioned such that the major axis of a first layer forms an oblique angle with respect to the major axis of a second layer, said oblique angle defining a long axis within the arc of the oblique angle.

34. The method of claim 33 wherein each additional layer is positioned such that the major axis of each additional layer is oriented at an angle different with respect to a long axis than the major axis of any of the previously contacted layers.

35. The method of claim 33 wherein each layer is individually prepared by electrospinning at least one fiber onto a rotating mandrel.

36. The method of claim 33 wherein at least one layer is directly electrospun onto at least one other layer.

37. The method of claim 35 wherein at least one layer is removed from the mandrel to yield a sheet of substantially aligned fiber and re-positioned with respect to at least one other layer.

38. The method of claim 33 wherein at least two layers are made to conform to a mold such that the long axis is circumferential to the center-line axis of the mold.

39. The method of claim 33 further comprising joining at least one fiber in each of two separate layers.

40. The method of claim 39 wherein said joining comprises chemical crosslinking

41. The method of claim 40 wherein said chemical crosslinking is photocatalyzed.

42. The method of claim 39 wherein said joining comprises applying adhesive, heat, pressure, microwave radiation, or combination thereof.

43. The method of claim 39 wherein said joining comprises growing tissue.

44. The method of claim 33 wherein at least one fiber is porogenic.

45. The method of claim 44 further comprising removing the porogenic fiber.

46. The method of claim 33 further comprising seeding the implant scaffold with at least one population of cells.

47. The method of claim 33 further comprising growing tissue onto or within the scaffold.

48. The method of claim 33 further comprising providing at least one therapeutic agent, biofactor, catalyst, or mixture or combination thereof.

49. The method of claim 33 further comprising incorporating a biocompatible hydrogel into the scaffold.

50. The method of claim 49 wherein the hydrogel comprise agarose, alginate, RGD-modified alginate, chitosan, collagen, fibrin, gelatin, hyaluronic acid, matrigel, oligo(poly(ethylene glycol)fumarate), poly(ε-caprolactone), poly(ethylene glycol), poly(glycolic acid), poly(glycolic-lactic acid), poly(lactic acid) or puramatrix.

51. The method of claim 49 wherein hydrogels are compatible with stem cells.

52. A kit containing a packaged sterilized (a) implant scaffold, said scaffold comprising at least two overlapping layers, each layer comprising at least one fiber aligned along a major axis of said layer, the layers being positioned such that the major axis of a first layer forms an oblique angle with respect to the major axis of a second layer, said oblique angle defining a long axis within the arc of the oblique angle; and (b) at least one support plate, insertion tool or adapter, carrier, or hydration jacket.

53. The kit of claim 52 comprising a pair of support plates, each positioned on opposing surfaces of the scaffold

54. A method of treating a mammalian patient comprising: (a) assessing the need to repair or replace at least one body part of said patient; (b) deciding that implanting a scaffold to facilitate the repair or replacement of said body part is a viable treatment for said patient; and (c) implanting into said patient an implant scaffold comprising at least two overlapping layers, each layer comprising at least one fiber aligned along a major axis of said layer, the layers being positioned such that the major axis of a first layer forms an oblique angle with respect to the major axis of a second layer, said oblique angle defining a long axis within the arc of the oblique angle.

55. The method of claim 54 wherein the body part comprises a biologic orthopedic or cardiovascular laminate.

56. The method of claim 55 wherein the body part comprises a intervertebral disc, a knee meniscus, a blood vessel, a tendon, a bladder wall, or a diaphragm.

57. The method of claim 54 wherein the patient is a human.

58. The method of claim 54 wherein the scaffold is attached to a bone, muscle, or tendon.
